(19) United States
(12) Patent Application Publication
(10) Pub. No.: US 2024/0428898 A1
(43) Pub. Date: Dec. 26, 2024

(54) System, Devices, and Methods for Three-Dimensional Analysis of Carbon Black

(71) Applicant: BIRLA CARBON U.S.A., INC., Marietta, GA (US)

(72) Inventors: Tyler C. Gruber, Marietta, GA (US); Katherine Cauffiel, Kennesaw, GA (US)

(21) Appl. No.: 18/693,988

(22) PCT Filed: Sep. 23, 2022
(86) PCT No.: PCT/US2022/044600

§ 371 Date: Mar. 21, 2024

Related U.S. Application Data
(60) Provisional application No. 63/247,327, filed on Sep. 23, 2021.

(51) Int. Cl. G16C 20/80; G06T 7/62; G06T 17/10; G16C 20/30; G16C 20/70; G16C 60/00

(52) U.S. Cl. CPC G16C 20/80; G06T 7/62; G06T 17/10; G16C 20/30; G16C 20/70; G16C 60/00; G06T 2207/10061

(57) ABSTRACT

Technologies are provided for three-dimensional (3D) reconstruction of carbon black aggregates and analysis of morphological properties based on reconstructed 3D aggregate models. Morphological properties determined for a carbon black aggregate of unknown type can be analyzed using a machine-learned predictive model that can identify a type of carbon black associated with the carbon black aggregate.

### CROSS-REFERENCE TO RELATED APPLICATION

This application claims the benefit of and priority to U.S. Provisional Patent Application No. 63/247,327, filed Sep. 23, 2021, the entire contents of which application are hereby incorporated herein by reference in their entireties.

### BACKGROUND

Two-dimensional (2D) transmission electron microscopy (TEM) has enabled connections between carbon black morphology and application properties. In turn, electron tomography-based three-dimensional (3D) aggregate reconstructions have revealed aggregate mass distribution. Yet, 3D characterization of carbon black has been hindered by various factors, some associated with characteristics of carbon black and others pertaining to the computational modeling involved in existing attempts to characterize carbon black morphology. For example, polydispersity of carbon black; the rather substantial wall-clock time commonly required for computational modeling of carbon black; and the ambiguous aggregate surfaces generated in 3D aggregate reconstruction have prevented accurate, statistically significant 3D characterization of carbon black morphology. Therefore, much remains to be improved in technologies for computational 3D analysis of carbon black morphology using electron microscopy images of carbon black aggregates.

### SUMMARY

It is to be understood that both the following general description and the following detailed description are illustrative and explanatory only and are not restrictive.

Embodiments of this disclosure include a computer-implemented method. The computer-implemented method includes identifying, using a two-dimensional (2D) transmission electron microscopy (TEM) image, a carbon black aggregate; and determining, based at least on an approximation to a volume of the carbon black aggregate, a number of spheres. The computer-implemented method also includes generating a three-dimensional (3D) aggregate model of the carbon black aggregate by determining a solution to an optimization problem with respect to an objective function based on sizes of the spheres and positions of the spheres within a defined volume, the solution representing the 3D aggregate model.

Additional elements or advantages of this disclosure will be set forth in part in the description which follows, and in part will be apparent from the description, or may be learned by practice of the subject disclosure. The advantages of the subject disclosure can be attained by means of the elements and combinations particularly pointed out in the appended claims.

This summary is not intended to identify critical or essential features of the disclosure, but merely to summarize certain features and variations thereof. Other details and features will be described in the sections that follow. Further, both the foregoing general description and the following detailed description are illustrative and explanatory only and are not restrictive of the embodiments of this disclosure.

### BRIEF DESCRIPTION OF THE DRAWINGS

The annexed drawings are an integral part of the disclosure and are incorporated into the subject specification. The drawings illustrate example embodiments of the disclosure and, in conjunction with the description and claims, serve to explain at least in part various principles, elements, or aspects of the disclosure. Embodiments of the disclosure are described more fully below with reference to the annexed drawings. However, various elements of the disclosure can be implemented in many different forms and should not be construed as limited to the implementations set forth herein. Like numbers refer to like elements throughout.

FIG. 1 illustrates an example of a process flow for 3D analysis of morphology of carbon black, in accordance with one or more embodiments of this disclosure.

FIG. 2 illustrates an example of an operating environment (including a computing system) for 3D analysis of morphology of carbon black, in accordance with one or more embodiments of the disclosure. The operating environment includes electron microscopy system and a computing system.

FIG. 3 is a TEM tilt image of several carbon black aggregates, where 63 carbon black aggregates are labeled with respective numerals. Select 30 aggregates are marked with respective closed curved lines to indicate that those aggregates pertain to a reconstruction and analysis group.

FIG. 4 illustrates an example of a determination of fitness metric for cost function at 0° projection angle for a reconstructed aggregate, in accordance with one or more embodiments of this disclosure.

FIG. 5 is a photographic projection of a 3D-printed assembly composed of 62 spherical bodies mounted on a rotation axis, in accordance with one or more embodiments of this disclosure. The 3D-printed assembly represents, at macroscopic scale, a model of a carbon black aggregate.

FIG. 6 illustrates a view of a 3D reconstruction of 3D-printed assembly illustrated in FIG. 5, in accordance with one or more embodiments of this disclosure.

FIG. 7A is a no-tilt (0°) TEM micrograph of a carbon black aggregate.

FIG. 7B is a no-tilt tomography view of the carbon black aggregate shown in FIG. 7A, as determined using conventional TEM tomographic reconstruction.

FIG. 7C is a no-tilt sphere-based 3D reconstruction view of the carbon black aggregate, in accordance with aspects of this disclosure.

FIG. 8 illustrates dependence of average reconstruction objective function on tilt angle interval and tilt angular range for 30 N330 carbon black aggregate reconstructions, in accordance with one or more embodiments of this disclosure.

FIG. 9 illustrates no-tilt (0°) TEM images, and 0° and 90° 3D aggregate model images of seven of the 30 select carbon black aggregates shown in FIG. 1. Images are not to the same scale.

FIG. 10 illustrates a distribution of converged average objective function values for 265 3D reconstructions for respective N330 carbon black aggregates, in accordance with one or more embodiments of this disclosure.

FIG. 11 illustrates a distribution of aggregate reconstruction times for 256 carbon black aggregates, in accordance with one or more embodiments of this disclosure.

FIG. 12 illustrates a distribution of 3D specific surface area (3DSSA) calculated from 256 3D reconstructions for respective N330 carbon black aggregates, in accordance with one or more embodiments of this disclosure. A subset of the 256 3D reconstructions corresponds to reconstructions of the select 30 N330 carbon black aggregates marked in FIG. 3.

FIG. 13 illustrates an anisometry metric (referred to “Relative Z”) as a function of relative 3D void volume (3DVV) for 30 N330 carbon black aggregate reconstructions, in accordance with one or more embodiments of this disclosure. Each reconstruction corresponding to a respective one of the 30 N330 carbon black aggregates marked in FIG. 3.

FIG. 14 illustrates 3D aggregate volume (from voxel count) versus 2D aggregate volume (from (8/3) A²/P) for 30 N330 carbon black aggregate reconstructions, in accordance with one or more embodiments of this disclosure. Each reconstruction corresponding to a respective one of the 30 N330 carbon black aggregates marked in FIG. 3.

FIG. 15 illustrates a distribution of specific surface area for 30 N330 carbon black aggregate reconstructions, in accordance with one or more embodiments of this disclosure. Each reconstruction corresponding to a respective one of the 30 N330 carbon black aggregates marked in FIG. 3.

FIG. 16 illustrates a relationship between 3DVV and 2DV′/V for 30 N330 carbon black aggregate reconstructions, in accordance with one or more embodiments of this disclosure. Each reconstruction corresponding to a respective one of the 30 N330 carbon black aggregates marked in FIG. 3.

FIG. 17 illustrates an example of process flow for 3D analysis and identification of carbon black, in accordance with one or more embodiments of this disclosure.

FIG. 18 illustrates an example of system for generation of a prediction model configured to identify a type of carbon black aggregate, in accordance with one or more embodiments described herein.

FIG. 19 illustrates an example of a an operating environment (including a computing system) to designate a carbon black aggregate as being of a particular carbon black, in accordance with one or more embodiments described herein.

FIG. 20 illustrates an example of a method in accordance with one or more embodiments of this disclosure.

FIG. 21 illustrates another example of a method in accordance with one or more embodiments of this disclosure.

FIG. 22 illustrates another example of a method in accordance with one or more embodiments of this disclosure.

FIG. 23 illustrates yet another example of a method in accordance with one or more embodiments of this disclosure.

FIG. 24 illustrates another example of a method in accordance with one or more embodiments of this disclosure.

FIG. 25 illustrates an example of computing system to implement 3D reconstruction and analysis of carbon black aggregates, in accordance with one or more embodiments of the disclosure.

FIG. 26 illustrates another example of a computing system to implement 3D analysis of carbon black aggregates, in accordance with one or more embodiments of this disclosure.

### DETAILED DESCRIPTION

This disclosure recognizes and addresses, among other technical challenges, the issue of three-dimensional reconstruction and analysis of carbon black morphology using electron microscopy images of carbon black. Embodiments of the disclosure, individually or in combination, advance the TEM-based elucidation of carbon black aggregate morphology by permitting the direct modelling of carbon black aggregate mass distribution in a statistically relevant way and on a timescale amenable to broad application in the laboratory. More specifically, embodiments of the disclosure provide computing systems, computing devices, computer-program products and computer-implemented methods for sphere-based tomographic reconstruction and analysis of carbon black aggregates. More specifically, embodiments of the disclosure provide a 3D reconstruction approach that relies on solving an optimization problem with respect to an objective function based on sizes and positions of a number of spheres within a defined volume. Such an approach yields 3D aggregate models that can be compactly defined in terms of a four-tuple for each sphere in the model, where the tuple includes a 3D position vector and a parameter defining a size of sphere. Embodiments of this disclosure, individually or in combination, can be applied to binarized TEM images of carbon black (CB) aggregates, resulting in complete descriptions of the spatial distribution of aggregate mass. Although embodiments of this disclosure are illustrated in connection with N330 CB aggregates, the disclosure is not limited in that respect. Indeed, the principles and practical applications of this disclosure in connection with 3D reconstruction and analysis of carbon black morphology can be directed to other types of carbon black and aggregates thereof—e.g., N110 carbon black, N220 carbon black, N330 carbon black, N550 carbon black, N660 carbon black, N772 carbon black, N880 carbon black, and N990 carbon black.

Embodiments of the disclosure yield 3D aggregate models that permit determining values of several morphological properties of carbon black aggregates. Not only are the values of the morphological properties consistent with existing 2D analysis of carbon black, but the availability of position and size of carbonaceous particles that form a carbon black aggregate permit more detailed analysis than in conventional tomographic reconstructions.

Embodiments of this disclosure are illustrated with 3D reconstructions based on a fixed number of spheres per carbon black aggregate and binary TEM images. The disclosed 3D sphere-based approach to reconstruction of carbon black aggregates, however, is not limited in that respect. Indeed, in some embodiments, 3D reconstruction of a carbon black aggregate can be based on a number of spheres that is treated as an optimization parameter and, thus, dynamically changes during the 3D reconstruction. Additionally, or in other embodiments, rather than using binary TEM images and corresponding modeled binary projections, the disclosed 3D sphere-based approach can be based on greyscale TEM images and corresponding simulated greyscale projections. Such modeled greyscale projections can be generated based on a suitable estimator of electron transmission intensity as a function of mass and/or gain/offset adjustments. In some cases, the estimator can be based on parameterized decaying exponential functions, where parameters that define the decaying functions can be used as optimization parameters in a 3D sphere-based reconstruction.

Further, although embodiments of this disclosure are illustrated with reference to spheres and carbon black, the principles and practical applications of this disclosure are not limited to either one of those. Indeed, the underlying mechanisms of imaging and approaches to 3D reconstruction described in this disclosure also can be applied to other structures of carbon, organic elements, or inorganic elements, using geometrical objects of non-spherical geometries in the modeling of those other structures in 3D. The imaging and approaches to 3D reconstructions can ultimately be applied to a material of any composition, where the material has a structure or several structures that can be modeled by groups of geometrical objects. As an example, curved tubular structures, such as single-wall carbon nanotubes (CNTs) or multi-wall CNTs, could be modeled as assemblies of right circular cylinders, each right circular cylinder described by seven parameters—three parameters indicative of a first position vector r corresponding to a first circular base of the right circular cylinder; three other parameters indicative of a position r′ of a second circular base of the right circular cylinder; and a single parameter indicative of the size of the first and second circular bases. In some cases, such curved tubular structures can be modeled as assemblies of truncated cones, each truncated cone described by eight parameters—three parameters indicative of a first position vector r corresponding to a first circular base of the truncated cone; three other parameters indicative of a position r′ of a second circular base of the right circular cylinder; and parameter indicative of the size of the first circular base; and a parameter indicative of the size of the second circular base. The approaches to 3D reconstruction in accordance with this disclosure are not limited to such non-spherical geometrical objects, and objects having other geometries also are contemplated.

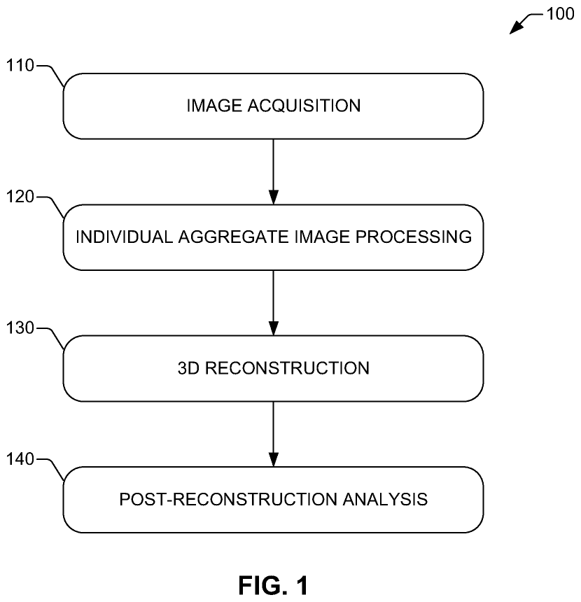

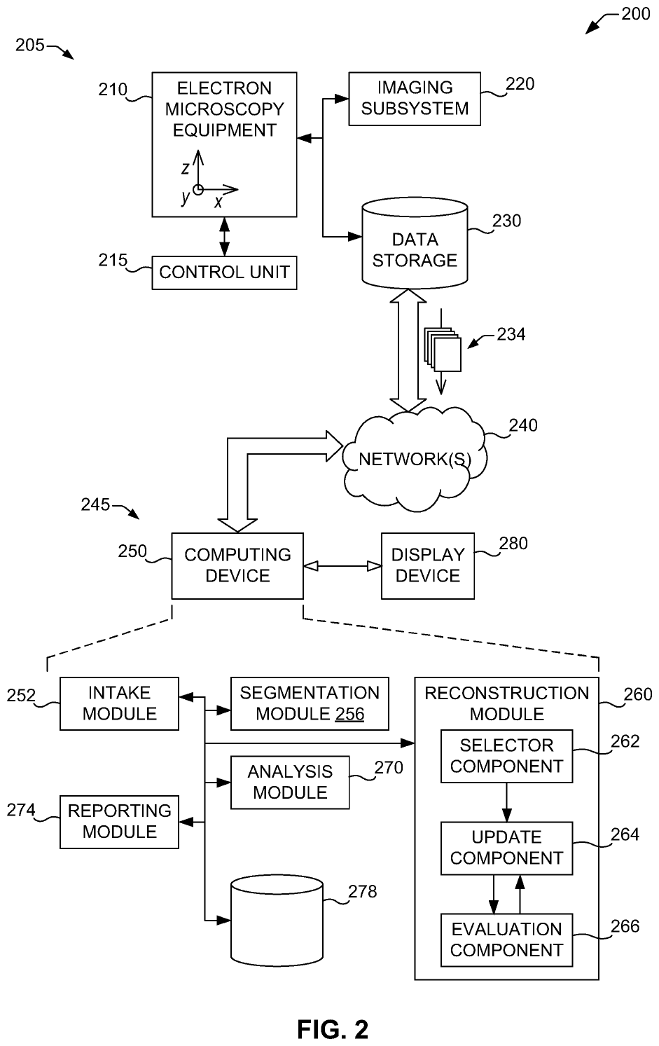

With reference to the drawings, FIG. 1 illustrates an example of a process flow 100 for 3D analysis of morphology of carbon black, in accordance with one or more embodiments of this disclosure. The process flow 100 includes multiple stages, represented by respective blocks in FIG. 1, that can be implemented individually or in combination with one another. The process flow 100 can be implemented, at least partially, in the operating environment 200 shown in FIG. 2.

TEM-based tomography involves radiation penetrating through a mass at several tilt angles about a rotation axis contained in a cross-sectional plane of the mass, and generating information-containing projection images onto that cross-sectional plane. Computed tomography, in turn, involves processing those projection images to create a 3D model of mass being probed. Resulting data can be referred to as a tomogram and define the 3D model as a density function of 3D vector positions (ρ(r)=ρ(x, y, z)). Accordingly, the process flow 100 includes an image acquisition stage 110 where imaging data defining TEM images of carbon black aggregates can be accessed. Those TEM images include a series of TEM 2D images of the carbon black aggregates, where each TEM 2D image is generated at a respective tilt angle. Each TEM 2D image in the series can be referred to as a TEM tilt image.

As is illustrated in FIG. 2, the series of TEM tilt images of carbon black aggregates can be generated by an imaging system 205. The imaging system 205 includes electron microscopy equipment 210 that can generate electric signals representing a distribution of carbon mass dispersed on a carbon film. The carbon mass forms multiple carbon black aggregates that can be dispersed ultrasonically in a solvent and then deposited on the carbon film (e.g., a glow-discharge-activated carbon film). To that end, the electron microscopy equipment 210 includes a source apparatus that serves as a source of electrons that can be injected into a collimator apparatus. The collimator apparatus can form a beam of electrons having nearly the same kinetic energy. Such a beam can be referred to as a monochromatic electron beam. The collimator apparatus can be elongated along an incidence direction (denoted by z in FIG. 2). From an output of the collimator apparatus, the monochromatic electron beam can enter a focusing apparatus within the electron microscopy equipment 210. The focusing apparatus can direct the monochromatic electron beam to a sample holder apparatus that also is part of the electron microscopy equipment 210.

The sample holder apparatus can include a TEM grid having the carbon film affixed thereto. The sample holder apparatus can be embodied in a goniometric stage (single-axis or multi-axis). Thus, a sample containing the carbon film can be rotated through a defined tilt angular range [−Θ, Θ], at defined consecutive tilt angle intervals Δθ. Such a rotation results in the sample being sequentially placed at a series of tilt angles through the defined tilt angular range. At each tilt angle, the sample can be subjected to the monochromatic electron beam. In some cases, Θ=70° and Δθ=2°, resulting in a series of 71 tilt angles. In some cases, Θ=70° and Δθ=10°, resulting in a series of 15 tilt angles. A control unit 215 can cause the goniometric stage to rotate through the defined tilt angular range [−Θ, Θ], at the defined consecutive tilt angle intervals Δθ. At each tilt angle, the control unit 215 also can cause the sample to be subjected to the monochromatic electron beam. To cause such rotations and exposure to the monochromatic electron beam, the control unit 215 can execute a control sequence having processor-executable instructions that, in response to being executed, cause the goniometric stage to rotate and the monochromatic electron beam to impinge on the sample. The control sequence can have further processor-executable instructions that, in response to being executed, cause the focusing apparatus to focus (or refocus) the electron beam prior to collecting each image.

The electron microscopy equipment 210 also can include a sensor apparatus that can detect scattered electrons that have passed through the TEM grid. In response to such a detection, the sensor apparatus can generate electric signals representing the arrangement of the carbon mass dispersed in the carbon film. The electric signals can be generated in sequence, according to the series of tilt angles within the defined tilt angular range [−Θ, Θ]. That is, electric signals are generated for each tilt angle in the series. As a result, batches of electric signals can be generated for respective tilt angles in the series of tilt angles.

The batches of electric signals can be passed individually, via a bus architecture, from the electron microscopy equipment 210 to an imaging subsystem 220. The imaging subsystem 220 can generate imaging data by processing a batch of electric signals generated at a tilt angle in the series of tilt angles. For that tilt angle, the imaging data define a TEM tilt image of the carbon black aggregates dispersed within the carbon film. By processing the batches of electric signals generated over the series of tilt angles, the imaging subsystem 220 can generate imaging data defining a series of TEM 2D images of the carbon black aggregates generated at respective tilt angles. The generation of such imaging data can be automated, via the control unit 215, by automating the rotation of the goniometric stage and subsequent exposure to the monochromatic electron beam. The automated generation of the imaging data can be referred to as automated image acquisition.

The imaging subsystem 220 can then retain the imaging data in one or multiple memory devices 230 (referred to as data storage 230). The imaging data can be retained in one or multiple files within a filesystem configured in the data storage 230. Each of the one or multiple files can be formatted according to suitable format for image storage.

As an illustration, automated image acquisition for a series of tilt angles through a [−70°,70°] range at Δθ=10° intervals can be performed in a fraction of an hour (from about 20 minutes to about 40 minutes, for example). Such a time period that is elapsed in the automated image acquisition can include short time intervals elapsed per respective images and longer time intervals interspersed between the short intervals. The longer time intervals correspond to respective setup periods prior to obtaining imaging data at a particular tilt angle. In some cases, each one of the short intervals can span approximately 30 seconds, and each one of the longer time intervals can span a few minutes. Thus, automated image acquisition can be substantially faster than in existing technologies for 2D analysis of carbon blacks. Accordingly, embodiments of this disclosure can efficiently acquire and store many Δθ=10° series of TEM tilt images. The acquisition and storage of multiple series of TEM tilt images can yield a wealth of immutable reusable imaging data that can be readily applied to any current or future reconstruction approaches.

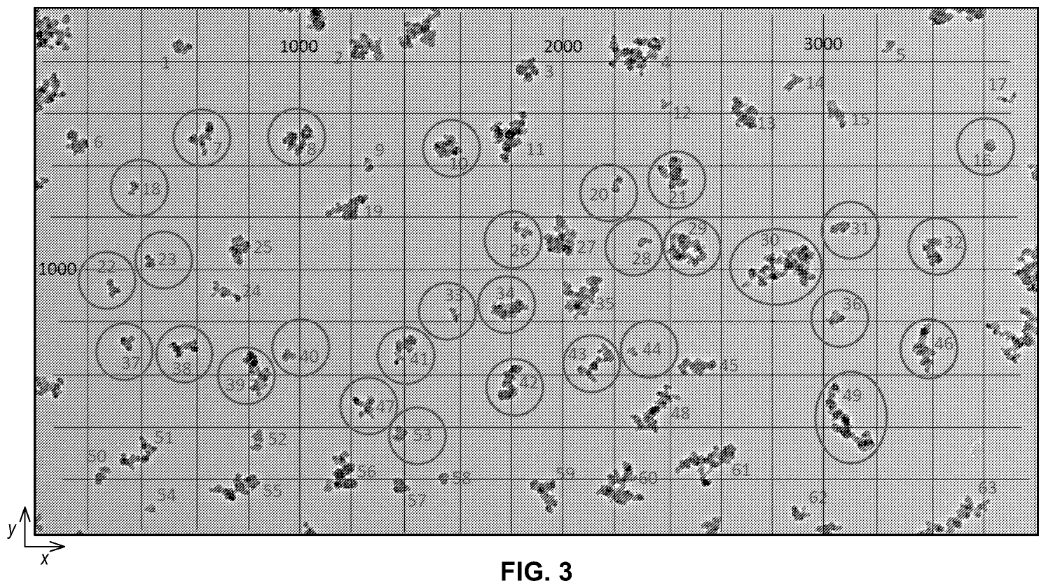

Simply for purposes of illustration, an N330 carbon black sample can be ultrasonically dispersed in chloroform and deposited on a glow-discharge-activated, carbon-filmed grid. The electronic microscopy equipment 210 can be used to generate a series of TEM tilt images of the aggregates of the N330 carbon black. To that end, the goniometric stage of the electron microscopy equipment 210 can rotate the sample through a [−70°, 70°] range at defined consecutive tilt angle intervals Δθ(2° or 10°, for example). Additionally, at each tilt angle, the sample can be subjected to a 120 kV monochromatic electron beam. Further, as mentioned, imaging data can be generated using electric signals generated by the sensor apparatus of the electron microscopy equipment 210. The sensor apparatus can include a charge-coupled device (CCD) having a 2672×4008 pixel array. Imaging resolution can be configured to be approximately 20 pixels per mean particle diameter, which corresponds to about 1.67 nm per pixel for N330 carbon black. That imaging resolution is about double the 10 pixels per mean particle diameter commonly used for 2D measurements of carbon black. Representative aggregate fields containing 50 to 100 aggregates can be selected for tomographic imaging and reconstruction in accordance with aspects of this disclosure. Of the 63 identified reconstructable aggregates in the first acquired series, 30 were chosen for initial reconstruction and method evaluation. As an example, FIG. 3 is the TEM tilt image corresponding to 0° tilt angle (referred to as zero-tilt) in the series of TEM tilt images. Select 30 carbon black aggregates used to illustrate various reconstruction and analysis aspects of this disclosure are individually marked with either a circle or an ellipse in FIG. 3. The select 30 carbon black aggregates are a subset of several reconstructable aggregates present in the series of TEM tilt images. Here, a reconstructable aggregate refers to a carbon black aggregate that is visible in all images in the series of TEM tilt images. A select carbon aggregate can be a reconstructable aggregate that satisfies a non-occlusion criterion dictating that the reconstructable aggregate does not occlude, or is not occluded by, another reconstructable aggregate in the field of view at any tilt angle through a defined tilt angular range [−Θ, Θ].

Generation of the TEM 2D images is one part of the image acquisition stage 110. Another part of the image acquisition stage 110 includes the transfer of imaging data from the imaging system 205, and can be implemented by a modeling system 245. More specifically, a computing device 250 that is part of the modeling system 245 can access a series of TEM 2D images 234 of carbon black aggregates generated at respective tilt angles. To that end, the computing device 250 can include an intake module 252 that can access, from the data storage 230, imaging data defining the series of TEM 2D images. Accessing the imaging data can include downloading the series of TEM 2D images programmatically from the data storage 230. For example, the intake module 252 can download the TEM 2D images via an application programming interface (API). By executing one or more function calls of the API, the intake module 252 can receive image files 234 corresponding to the series of TEM 2D images, and can retain the files 234 within one or more non-volatile memory devices 278 (referred to as memory 278). As another example, the intake module 252 can download the TEM 2D by executing a script to copy the image files 234 from the filesystem within the data storage 230 to the memory 278. The memory 278 can be integrated into the computing device 250. The computing device 250 can include computing resources (not shown) comprising, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources.

Each TEM tilt image of the carbon black aggregates depicts multiple carbon black aggregates. As is illustrated in FIG. 3, the depicted carbon black aggregates can be well separated and readily distinguishable from one another. The process flow 100 can include an aggregate image processing stage 120 that permits segmenting a TEM tilt image into multiple TEM tilt images depicting respective carbon black aggregates. As such, for a series of TEM tilt images, the aggregate image processing stage 120 can permit identifying individual carbon black aggregates within a TEM tilt image in the series and generating multiple other series of TEM tilt images, with each one of the multiple other series corresponding to one respective carbon black aggregate. In other words, implementation of the image processing stage 120 can yield multiple series of TEM tilt images for a number Q of carbon black aggregates, where a first series of the multiple series of TEM tilt images corresponds to a first carbon black of the Q carbon black aggregates; a second series of the multiple TEM tilt images corresponds to a second carbon black of the Q carbon black aggregates; continuing up to a j-th series of the multiple series of TEM tilt images for a j-th carbon black aggregate of the Q carbon black aggregates; and further continuing up to a Q-th series of the multiple series of TEM tilt images for a Q-th carbon black aggregate of the Q carbon black aggregates. Simply as an illustration, for carbon black aggregate no. 21 in FIG. 3, implementation of the aggregate image processing stage 120 can yield a series of TEM tilt images for that particular carbon black aggregate. Additionally, continuing with the illustration, for carbon black aggregate no. 30 in FIG. 3, implementation of the aggregate image processing stage 120 can yield a series of TEM tilt images for that particular carbon black aggregate.

The modeling system 245, via the computing device 250 (FIG. 2), also can implement the aggregate image processing stage 120. The computing device 250 can include a segmentation module 256 that can identify a particular carbon aggregate within a TEM tilt image and can then extract a section of the TEM tilt image, the section depicting the particular carbon aggregate and, optionally, a portion of image background. That portion of image background corresponds to areas of the TEM tilt image that surround the carbon black aggregate and lack carbon mass. The segmentation module 256 can identify the particular carbon black aggregate by applying one or more machine-vision techniques to determine pixels indicative of a periphery of the particular carbon black aggregate. Extracting that section of the TEM tilt image can include selecting imaging data defining values of pixels within, and including, that periphery. The imaging data can optionally define values of pixels indicative of surrounding areas of the carbon black aggregate. The machine-vision technique(s) can include a template-matching technique for object-localization as is described herein.

The segmentation module 256 can identify a particular carbon black aggregate across a series of TEM tilt images corresponding to respective tilt angles. To that end, in some cases, the segmentation module 256 can apply a template-matching technique to the series of TEM tilt images. Applying the template matching technique can include generating a template (also referred to as a feature) for the particular carbon black at first TEM tilt image of the series of the TEM tilt images. In one example, the first TEM tilt image can be the no-tilt TEM image in the series, such as FIG. 3. Here, for purposes of illustration, a template (or feature) of a carbon black aggregate refers to a data structure identifying a collection of pixels in a binary TEM tilt image of a carbon black aggregate, where the pixels in that collection are adjacent to one another. Applying the template-matching technique also includes comparing the template for the particular carbon black aggregate to pixels within a defined search region in a next TEM tilt image of the series of TEM tilt images. Continuing with the example, the next TEM tilt image can correspond to a tilt angle Δθ.

Further, because the particular carbon black aggregate is the aggregate being searched across TEM tilt images, the defined search region can be a rectangular area having a base (or first side) that includes the position of the particular carbon black aggregate along the tilt axis (x axis in FIG. 3, for example) in the field of view, and spans a limited portion of the entire field of view along the tilt axis. That position of the particular carbon black aggregate along the tilt axis may be the position of the particular carbon black aggregate in the first TEM tilt image. The rectangular area can span the entire length of the field of view along the axis that is normal to the rotation axis. In FIG. 3, the rotation axis is the x axis and the other axis is they axis. By confining the defined search region in such a fashion, the search for, and identification of, the particular carbon black aggregate in the next TEM tilt image can be computationally efficient. Comparing the template to pixels in the search region can include comparing pixels in the binarized version of the next TEM tilt image to the template. The segmentation module 256 can identify the particular carbon black aggregate in such a binarized image in response to determining that a threshold number of pixels correspond to (or match) the template. Other matching criteria besides threshold number of corresponding pixels also can be contemplated. After identifying the particular carbon black aggregate in the binarized version of the next TEM tilt image, the segmentation module 256 can generate an individualized TEM tilt image for the carbon black aggregate at the tilt angle of the next TEM tilt image by cropping a portion of the next TEM tilt image, the portion depicting the carbon black aggregate.

Further, the segmentation module 256 can continue searching for the particular carbon black aggregate in a subsequent next TEM tilt image in the series of TEM tilt images. Continuing with the example above, the subsequent next TEM tilt image can correspond to a tilt angle 2Δθ. Because the position of the particular carbon black aggregate along the tilt axis (x axis in FIG. 3) may remain unchanged upon rotation about that axis, the segmentation module 256 can confine the search to the same rectangular area used in prior searches. Rather than utilizing the template defined at the first TEM tilt image in the search for the carbon black aggregate at the subsequent next TEM tilt image, the segmentation module 256 can determine a next template for the particular carbon black aggregate based on an immediately prior TEM tilt image where the particular carbon black aggregate has been identified. That is, rather than applying a common template across the series of TEM tilt images, the segmentation module 256 can update a template for each adjacent pair of TEM tilt images. In other words, for a tilt TEM image at a defined tilt angle, the segmentation module 256 applies template matching using a template τ, and for a next tilt TEM image at a consecutive defined tilt angle, the segmentation module 256 updates from the template τ to a template τ′, and then uses the updated template τ′ in the next tilt TEM image. After identifying the particular carbon black aggregate in the binarized version of the subsequent next TEM tilt image, the segmentation module 256 can generate an individualized TEM tilt image for the carbon black aggregate at the tilt angle of the subsequent next TEM tilt image (e.g., 2Δθ) by cropping a portion of the next TEM tilt image, the portion depicting the carbon black aggregate.

In some cases, instead of utilizing a rectangular area as search region across TEM tilt images in the series of TEM tilt images, additional computation efficiency can be gained by searching in another rectangular area that is more confined. That other confined rectangular area also has a base (or first side) that includes the position of the particular carbon black aggregate along the tilt axis (x axis in FIG. 3, for example) in the field of view, and spans a limited portion of the entire field of view along the tilt axis. Additionally, that other rectangular area can have a height (or second side) that includes the anticipated position of the particular carbon black on the axis normal to the rotation axis for a defined tilt angle corresponding to the TEM tilt image where the particular carbon black aggregate is being searched. The segmentation module 256 can determine the anticipated position by multiplying the position of the particular carbon black aggregate at the zero-tilt TEM tilt image by a factor equal to the cosine of the defined tilt angle. Multiplying by such a factor can account for the positional changes of the particular carbon black aggregate within the field of view upon a rotation about the tilt axis.

Other defined search regions can be utilized to search for the particular carbon black aggregate. In some cases, respective defined search regions can be defined for each TEM tilt image in the series of TEM tilt images. Each one of the respective defined search regions can be defined relative to a reference carbon black aggregate that is common across the series of TEM tilt images. More specifically, the reference carbon black aggregate can be previously identified, also using template matching for example, in each one of the series of TEM tilt images. As such, for each TEM tilt image of the series of TEM tilt images, the segmentation module 256 can determine the position of the reference carbon black aggregate. The position of the reference carbon black aggregate in a TEM tilt image of the series of TEM tilt images can serve as a reference position for a defined search region in that TEM tilt image. In particular, the segmentation module 256 can configure that reference position as the geometrical center of the defined search region for that TEM tilt image. Additionally, the defined search region can be a rectangular area centered at the configured geometrical center, and can have a defined first side (or base) and a defined second side (or height). Placement of the respective defined search regions can thus vary across the series of TEM tilt images. In addition, from TEM tilt image to TEM tilt image in the series of TEM tilt images, the segmentation module 256 can adjust the size of the defined second side for a current image by multiplying the size of the second side in a prior image by a factor equal to the cosine of the defined tilt angle corresponding to the current image. After the segmentation module 256 has defined a search region with respect to the reference carbon black aggregate in a TEM tilt image of the series of TEM tilt images, the segmentation module 256 can search, and identify, the particular carbon black aggregate in that TEM tilt image using template matching in accordance with aspects described herein.

Various computational efficiencies can be accomplished by using search regions defined relative to the reference carbon black aggregate. For example, defining a search region relative to the carbon black aggregate can readily account for changes that may occur in the position of the particular carbon black aggregate along the tilt axis (x axis in FIG. 3) across two or more images of the series of TEM tilt images.

As a result of applying the template-matching technique, or other object-localization techniques, for each one of the TEM tilt images in the series of TEM tilt images, the segmentation module 256 can generate imaging data defining an individualized TEM tilt image corresponding to a section containing the particular carbon black aggregate. As a result, the segmentation module 256 can generate a series of individualized TEM tilt images corresponding to respective tilt angles and depicting the particular carbon black aggregate in isolation from other carbon black aggregates in the TEM tilt images. Imaging data defining the series of individualized TEM tilt images can be retained within the memory 278. The segmentation module 256 can repeat the application of the template-matching technique described herein in order to identify other carbon black aggregates across the series of TEM tilt images corresponding to respective tilt angles. As a result, as mentioned, the segmentation module 256 can generate several series of individualized TEM tilt images, each one of the series corresponding to one respective carbon black aggregate.

Because the segmentation module 256 can identify a particular carbon black across a series of TEM tilt images by applying machine-vision techniques, pre-alignment of the aggregate images was not necessary. Accordingly, in sharp contrast to existing technologies, fiducial markers are unnecessary in embodiments of this disclosure. Fiducial markers are opaque objects that can be placed in the field of view of an electron microscope, and are commonly used in traditional tomography to aid alignment. TEM image acquisition and processing of this disclosure can be simplified relative to existing technologies.

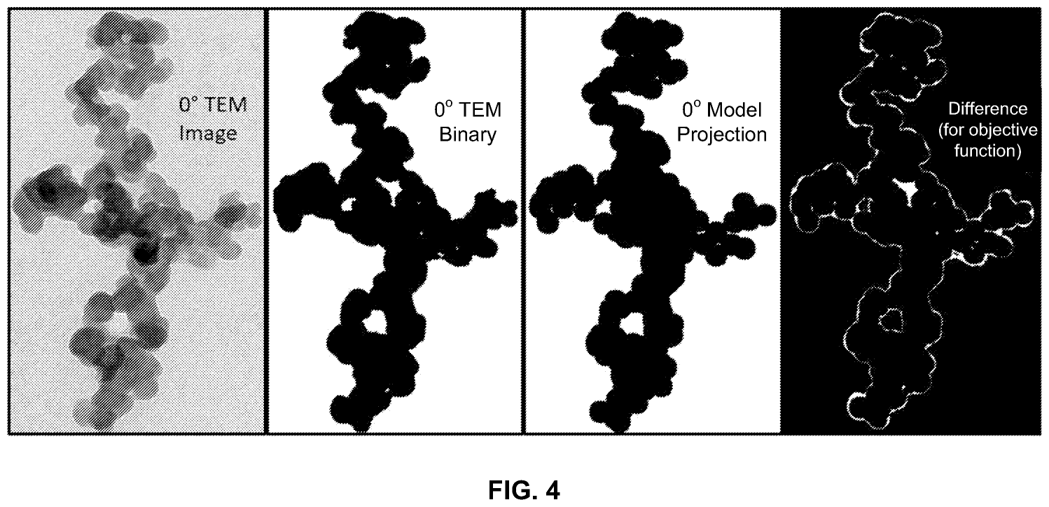

For a select carbon black aggregate, the segmentation module 256 also can generate a binarized image for each TEM image in a series of individualized TEM tilt images corresponding to the select carbon black aggregate. Accordingly, for each TEM tilt image in the series, the segmentation module 256 can transform the TEM tilt image in greyscale into a binary TEM image. As an illustration, FIG. 4 presents a 0° TEM image (in greyscale) and the corresponding 0° binary image. As a result, the segmentation module 256 can generate a series of binarized TEM tilt images for the select carbon black aggregate. The segmentation module 256 can retain imaging data defining the series of binarized TEM tilt images within the memory 278. The segmentation module 256 can generate a binarized image by applying one of numerous techniques for 2D automated image analysis. Specifically, an example technique includes, for each TEM tilt image (in greyscale) in the series of individualized TEM tilt images, (i) smoothing the TEM tilt image by applying a median filter with a defined radius (e.g., 3×3 pixels (or Radius 1)). The example technique also includes (ii) flattening the resulting TEM tilt image (still in greyscale) by applying a rolling-ball algorithm. The flattening can level the background of the image, and can cause filling of valleys, without cutting peaks. Existing 2D image processing techniques to binarize a greyscale image typically lack such background leveling. The example technique further includes (iii) applying an autothreshold of a first type (e.g., IJ_IsoData) to the flattened TEM tilt image (still in greyscale) and also applying an autothreshold of a second type (e.g., Triangle) to the flattened TEM tilt image. The application of the autothreshold of the first and second types yield respective threshold values. Existing 2D image processing techniques to binarize a greyscale image typically provide simpler autothreshold approaches. (iv) The example technique still further includes generating a binary TEM tilt image by applying an average of the respective threshold values to the flattened TEM tilt image. Application of the average of the respective threshold values results in the assignment of some grey tones (or pixel values) in the flattened TEM tilt image to a pixel value indicative of black, and the assignment of other grey tones (or pixel values) in the flattened TEM tilt image to a pixel value indicative of white. Accordingly, each pixel value in the flattened TEM tilt image is transformed to either black or white, yielding the binary TEM tilt image. The example technique also includes (v) applying a morphological “open” operation—a combination of an erode operation and then a dilate operation—to the binary TEM tilt image. The resulting binary image is configured as the binarized TEM tilt image TEM tilt image in greyscale. The thickness of pixel skin that is removed in the “open” operation can be configurable. The number of adjacent background pixels necessary before a pixel is removed from edge of an object during the erode operation also can be configurable. The number of adjacent foreground pixels necessary before a pixel is added to the edge of the object during dilate operation also can be configurable.

The process flow 100 (FIG. 1) also includes a 3D reconstruction stage 130 where a 3D aggregate model of a carbon black aggregate can be generated. The modeling system 245, via the computing device 250 (FIG. 2), can implement the 3D reconstruction stage 130. The computing device 250 can include a reconstruction module 260 that includes a selector component 262 that can identify, based on a TEM 2D image of a carbon mass, a particular carbon black aggregate to be reconstructed. The selector component 262 can then determine, using the TEM 2D image, a number of spheres N for 3D reconstruction of the particular carbon black aggregate. Each sphere represents a carbonaceous particle that is part of the particular carbon black aggregate. The TEM 2D image can be a no-tilt (0°) TEM tilt image in a series of TEM tilt images of the carbon mass. The number of spheres N can be defined as the 2D-based estimate of number of particles in the aggregate (NPA). Hence, N can be determined as the quotient of volume of the aggregate (V_{agg}) divided by particle volume (Vₚₐᵣₜ). That is, N=V_{agg}/Vₚₐᵣₜ. Here, V_{agg} can be determined using the aggregate volume estimator (8/3)A²/P, which is an approximation of an area and perimeter relationship derived for random flocs, where A denotes area of the aggregate and P denotes perimeter of the aggregate. Additionally, Vₚₐᵣₜ can be determined from a particle size mean chord model. More specifically, the particle size can be represented by a diameter d of spherical particle, where d is estimated from A and P as follows: d=απ(A/P). The parameter α is equal to the greater of 13.092(P²/A)^{-0.92} or 0.4. The particle volume can then be determined using Vₚₐᵣₜ=πd³/6. Accordingly, to determine N, the selector component 262 can apply 2D analysis to the TEM 2D image to determine V_{agg} and Vₚₐᵣₜ for the particular carbon aggregate.

After N has been determined, the reconstruction module 260 can generate the 3D aggregate model of the particular carbon black aggregate by determining a solution to an optimization problem with respect to an objective function based on sizes of the N spheres and positions of the N spheres within a defined volume in three-dimensional space. The determination of such a solution can be achieved via an iterative process that, in response to converging or satisfying another type of termination criterion, yields model data defining the 3D aggregate model. Upon convergence, the iterative process also yields a converged value of the objective function. Each iteration in that process can be referred to as a reconstruction step.

An update component 264 can update an arrangement of the N spheres within a defined volume. The arrangement is defined by a configuration of positions and a configuration of sizes of the number of spheres within the define volume. Thus, updating the arrangement can include updating the configuration of positions and updating the configuration of sizes. In other words, updating the arrangement can include adjusting size and position of each one of the N spheres. In some cases, an update approach can include updating a single sphere at each reconstruction step, cycling in order through the spheres as reconstruction steps are performed. Specifically, at iteration j (or j-th reconstruction step), position of a single sphere τ_{λ}(λ being an index equal to one of 1, 2. . . N−1, or N) of the N spheres can be updated while position and sizes of the other N−1 spheres remain unchanged. Subsequently, at iteration j+1, position of a single sphere τ_{κ+1} of the N spheres can be updated while position and sizes of the other N−1 spheres remain unchanged at that next iteration. As iterations continue, each one of the spheres may have been individually updated, in turn, such that at iteration j+N+1, sphere τ_{λ} is again updated, continuing to traverse the set of spheres as next iterations are implemented and the position of other spheres are individually updated in turn. An update to the position of a sphere can be implemented in several ways. As an illustration, the position of the sphere being updated can be represented by a position vector r that can be defined in a Cartesian coordinate system; thus, r can be the triple (x,y,z). Updating the position of that sphere can include modifying x to x+Δx, y to y+Δy, and z to z+Δz. Here, Δρ=XD (ρ=x, y, z) where D is a displacement value and λ is a uniform random number in the interval [0,1]. In some cases, the displacement value D=α [symbol] d [symbol] , where α is a parameter that decays exponentially from a maximum value to a minimum value as iteration j increases, and [symbol] d [symbol] is the average diameter of the N spheres at a prior iteration. Because α decays as iterations progress, larger displacements—and ensuing coarser position updates—are implemented earlier in the iterative process to determine a solution to the optimization problem, and subsequent smaller displacements—and ensuing finer position updates—later in the iterative process. Such early coarser position updates followed by smaller displacements (or finer position updates) can permit mitigating or avoiding local minima of the objective function. Further, based on the iteration j, size of that single sphere also can be updated. For example, size of a sphere can begin to be modified after a defined number of reconstruction steps have been implemented. A magnitude of a modification to diameter of the sphere can be decayed exponentially as iteration j increases. Other approaches to updating the arrangement of sphere can be implemented. For example, updating that arrangement can include performing a simulated-annealing update, performing a genetic algorithm update, or performing a conjugate-gradient steepest descent update.

The selector component 262 can obtain a binarized TEM image of the particular carbon black aggregate. The binarized TEM image can be retained in memory 278, and corresponds to an observed binarized TEM tilt image at a defined tilt angle. The observed binarized TEM tilt image can be one of a series of individualized binarized TEM tilt images over a tilt angular range [−Θ,Θ], at consecutive tilt angle intervals Δθ. In one example, Θ=70° and Δθ=10°, and the defined tilt angle pertains to the set {−70°, −60°, −50°, −40°, −30°, −20°, −10°, 0°, 10°, 20°, 30°, 40°, 50°, 60°, 70°}.

The selector component 262 can pass the binarized TEM image to an evaluation component 266 included in the reconstruction module 260. The evaluation component 266 can then determine a projection of the arrangement of the N spheres onto a plane corresponding to the defined tilt angle. In addition, the evaluation component 266 also can determine a fitness metric based on the binarized TEM image and the projection. The fitness metric can be the number of mismatched pixels (or, in some cases, a function of that number) resulting from a pixel-wise difference between the binarized TEM image and the projection. That is, the number of carbon black pixels and background pixels that are different in the projection relative to, pixel-wise, carbon black pixels and background pixels in the binarized TEM image. FIG. 4 illustrates an example of the pixel-wise difference for a 0° tilt TEM image.

The selector component 262 can traverse the series of individualized binarized TEM tilt images, passing each one of the binarized TEM tilt images to the evaluation component 266 for determination of a fitness metrics at the defined tilt angle of the binarized TEM tilt image that has been received by the evaluation component 266.

After the series of individualized binarized TEM tilt images has been traversed, the evaluation component 266 can update an objective function based on an average of the fitness metric over the series of individualized binarized TEM tilt images. In some cases, as the 3D reconstruction proceeds, the evaluation component 266 can determine that a value of the objective function after the update is unsatisfactory. In response, the evaluation component 266 can direct the update component 264 to update the current arrangement of the N spheres. In other words, the evaluation component 266 can cause the implementation of another reconstruction step, including the evaluation of updated projections based on the updated arrangement. In some cases, also in response to the objective function after the update being unsatisfactory, the evaluation component 266 can direct the update component 264 to reject the current arrangement of the N spheres and to revert to an immediately prior arrangement of the N spheres before a next update to the arrangement of the N spheres is implemented. By rejecting a current arrangement of the N spheres in scenarios where a value of the objective function for such an arrangement is unsatisfactory, the reconstruction module 260 may mitigate, or even avoid altogether, exploring paths in a hypersurface of the objective functions that do not lead to optimization (e.g., minimization) of the objective function. As a result, such a rejection of arrangement(s) of the N spheres can yield more efficient utilization of computing resources, such as processing time (or number of processor cycles) and/or memory storage.

After the implementation of a number of reconstructions steps, the evaluation component 266 can determine that a value of the objective function is satisfactory. To that end, the evaluation component 266 can determine that a value of the objective function satisfies a convergence criterion. The convergence criterion can dictate that magnitude of a difference between a current value of the objective function and a prior value in an immediate prior determination of the objective function is less than or equal to a threshold value. In response to determining that the value of the objective function is satisfactory—that is, the iterative 3D reconstruction process has converged—the evaluation component 266 can terminate the 3D reconstruction and can configure a current model (obtained after the last update to the arrangement of N spheres) as a 3D aggregate model of the particular carbon black aggregate. The evaluation component 266 can optionally terminate the iterative 3D reconstruction process even in the absence of convergence. To that end, the evaluation component 266 can optionally determine if a termination criterion is satisfied. The termination criterion can be a threshold number Nₘₐₓ of updates have been performed, for example. In some cases, the threshold number Nₘₐₓ can be defined as a multiple of the number of spheres (N) in the arrangement of spheres—that is Nₘₐₓ=qN where q is a natural number (e.g., 40, 50, or 60). After Nₘₐₓ updates (or iterations), changes to the arrangement of the spheres can cease, and the iterative 3D reconstruction process can end. In one example, q=50, and for N=60, the iterative 3D reconstruction process can thus end upon completing 3000 reconstruction steps. By introducing a termination criterion, the amount of computing resources may be bound, thus avoiding inefficient use of those resources (e.g., processor time and/or memory usage).

The 3D aggregate model of the carbon black aggregate that is generated by applying the iterative reconstruction approach described herein includes three-dimensional vector positions and sizes (e.g., diameters or radii) of each constituent sphere in the 3D aggregate model. That is, for each sphere Σ_{κ}(κ=1, 2 . . . N−1, N) the 3D aggregate model can include a four-tuple (r_{κ}; ν_{κ}) where r_{κ} is a position vector in 3D space and σ_{κ} is a parameter indicative of a size of the sphere Σ_{κ}. The parameter can define the diameter or radius of the sphere Σ_{κ}. Vector positions can be defined in an orthogonal coordinate system. For example, position vectors can be defined in a Cartesian coordinate system, and thus, r_{κ} can be the triple (x_{κ},y_{κ},z_{κ}). The reconstruction module 160 can retain such data defining the 3D aggregate model in the memory 278.

Such a compact form of the data defining the 3D aggregate model provides an improvement of the 3D reconstruction approach of this disclosure relative to conventional tomography. Indeed, for sphere-based 3D aggregate reconstruction, four parameters per sphere yields on the order of 10 to 1000 parameters for commonplace carbon black aggregates, as opposed to 10,000 or more parameters to record surface voxel coordinates derived from conventional reconstructions. Accordingly, embodiments of this disclosure utilize computing resources, such as memory storage, more efficiently that existing 3D reconstruction technologies.

Although the iterative reconstruction approach described hereinbefore relies on a same series of binarized TEM tilt images for each reconstruction step, the disclosure is not limited in that respect. Indeed, in some embodiments, after a defined number of reconstruction steps has been implemented, a current series of binarized TEM tilt images can be updated to create another series of binarized TEM tilt images. After such an update, subsequent reconstruction steps can be implemented using that other series of binarized TEM tilt images until the defined number of reconstructions steps has again been implemented. At that point in the iterative reconstruction approach, that other series of binarized TEM tilt images can be updated to generate a next series of binarized TEM tilt images. Subsequent reconstruction steps can be implemented using the next series of binarized TEM tilt images. Those successive updates can continue until the iterative reconstruction approach is terminated.

More specifically, in some embodiments, the reconstruction module 260 and the segmentation module 256 can update a current series of binarized TEM tilt images of a carbon black aggregate. The reconstruction module 260, via the evaluation component 266, for example, can determine if the current series of binarized TEM tilt images is to be updated. The current series of binarized TEM tilt images corresponds to an observed series of TEM tilt images for the carbon black aggregate. To that end, the reconstruction module 260 can determined if an update criterion is satisfied. The update criterion can be that a defined number of reconstructions steps have been performed. Other update criteria also can be contemplated. In some instances, the reconstruction module 260 can determine that the update criterion is satisfied and, as a result, the reconstruction module 260 can obtain a binarized TEM image of the carbon black aggregate at a defined tilt angle. The binarized TEM image is part of the current series of binarized TEM tilt images and, thus, corresponds to a defined tilt angle. The reconstruction module 260 also can determine or otherwise generate a projection of an arrangement of N spheres representing a current 3D model of the carbon black aggregate. Such an arrangement can be defined by a set of current four-tuples {(r_{κ}; σ_{κ})} for respective spheres Σ_{κ}(κ=1, 2 . . . N−1, N). The current four-tuples do not represent an optimized or otherwise converged 3D model of the carbon black aggregate.

In some embodiments, the reconstruction module 260, via the update component 264, for example, can apply a displacement to the binarized TEM image. In other embodiments, the reconstruction module 260 can direct the segmentation module 256 to apply such a displacement. Regardless of the module that applies the displacement, the displacement can be represented by a displacement vector d pertaining to a set of multiple displacement vectors. A definition of the set of multiple displacement vectors can be retained in the memory 278, for example. In some cases, each displacement vector of the set of multiple displacement vectors can be a point within a two-dimensional square lattice spanning (2N_{L}+1)×(2N_{L}+1) pixels, where NL is a natural number. In one example, N_{L}=5 and 121 displacement vectors form the set of multiple displacement vectors. Such a displacement vector d can be defined in a Cartesian system of coordinates having an origin at the geometrical center of the square lattice. Thus, the displacement vector can be represented as a pair (nₓ, n_{y}), where nₓ and n_{y} are each an integer number in the interval [−N_{L},N_{L}]. The geometrical center is located at a pixel, which pixel can be referred to as center pixel, simply for the sake of nomenclature. As such, applying the displacement can include displacing the binarized TEM image by the displacement vector representing the displacement. Applying the displacement results in a displaced binarized TEM image. In cases where the segmentation module 256 applies the displacement, the reconstruction module 260 can receive imaging data defining the displaced binarized TEM image. Regardless of how the displacement is applied, the reconstruction module 260 can retain the resulting displaced binarized TEM image within the memory 278, in a filesystem therein, for example.

The reconstruction module 260, via the evaluation component 266, for example, can determine a fitness metric based on the displaced binarized TEM image and the projection. As is described herein, the fitness metric can be the number of mismatched pixels (or, in some cases, a function of that number) resulting from a pixel-wise difference between the binarized TEM image and the projection. Thus, determining the fitness metric can include determining the number of mismatched pixels. The reconstruction module 260 can update a data structure to add the fitness metric that has been determined. The data structure can be retained in the memory 278.

The reconstruction module 260 can continue traversing the set of multiple displacement vectors to determine fitness metrics for respective displacements applied to the binarized TEM image of the carbon black aggregate at a defined tilt angle. After a fitness metric is determined, the reconstruction module 260 can update the data structure retained in the memory 278 in order to add the fitness metric that has been determined. After the set of multiple displacement vectors has been traversed fully, the reconstruction module 260 can identify or otherwise determine a displaced binarized image corresponding to a satisfactory fitness metric. To that end, the reconstruction module 260 can determine, using the data structure, the satisfactory metric, and can then identify and/or select the displaced binarized image from the memory 278. Because each fitness metric that is determined during traversal of the set of multiple displacement vectors is indicative of a number of mismatched pixels, a satisfactory fitness metric can be indicative of the least number of mismatched pixels. That is, the displaced binarized image that is identified can have the least pixel mismatch relative to the projection.

The reconstruction module 260, via the update component 264, for example, can update a next series of binarized TEM tilt images to incorporate the displaced binarized image that has been identified. Updating the next series of binarized TEM tilt images can include generating such a series. As other TEM tilt images (binarized or otherwise) are identified, the reconstruction module 260 can update the next series of binarized TEM tilt images at the memory 278. Accordingly, the next series of binarized TEM tilt images can be formed by displaced binarized images that each minimize the pixel mismatch with the projection, at the defined angle, of the current 3D aggregate model of the carbon black aggregate.

The reconstruction module 260 can continue traversing the current series of binarized TEM tilt images, applying the set of multiple displacement vectors to each image current series of binarized TEM tilt images. After the current series has been traversed, the reconstruction module 260 can configure the next series of binarized TEM tilt images as the current series of binarized TEM tilt images. Additional reconstructions steps can then be implemented as is described herein.

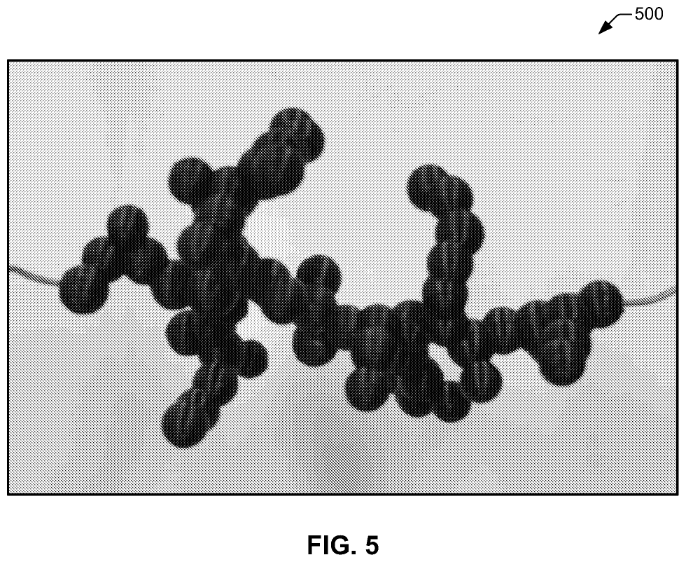

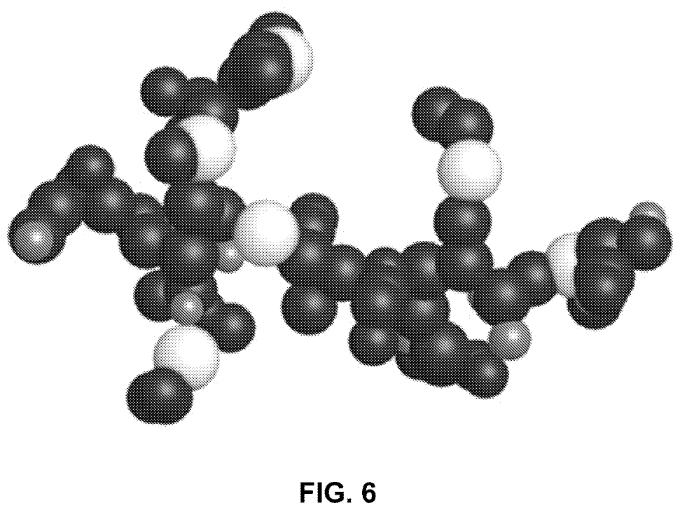

In order to illustrate the validity of the 3D reconstruction approach of this disclosure, the reconstruction module 260 can generate a 3D aggregate model of a 3D-printed assembly of 62 overlapping spheres. Such an assembly represents, at macroscopic scale, the structure of a carbon aggregate. Accordingly, the 3D aggregate model in such a case can be referred to as a 3D reconstruction model. FIG. 5 illustrates a photographic projection of the 3D-printed assembly mounted on a rotational axis. Five other photographic projections are determined for the 3D-printed assembly and used to create a series of binary images. FIG. 6 illustrates a 3D reconstruction model view corresponding to the photographic projection shown in FIG. 5. In FIG. 6, tone of grey shading of a sphere indicates a range of diameter, where the range is defined in terms of multiples of mean particle diameter d. Thus, spheres having a diameter within a particular range can be depicted as having slightly different size, but same tone of grey shading. Darker shading tone indicates lesser range of diameter. The resulting average objective function value of the 3D reconstruction was 18%. Without intending to be bound by interpretation and/or conjecture, such value may arise in part from tilt angle inaccuracies, slight x-y plane rotational misalignment, and possible projection distortions.

Further, the segmentation module 256 can generate, based on sphere data outputs from the 3D-printed assembly, simulated binary images for the sake of validation of the 3D reconstruction approach of this disclosure. In one example, a series of 15 simulated binary tilt images obtained for a [−70°,70°] tilt angular range and Δθ=10° is provided to the 3D reconstruction approach. Average objective function values upon convergence (or otherwise termination) of the 3D reconstruction approach for five separate trial reconstructions ranged from 2.3% to 3.1%. A trial reconstruction is a 3D reconstruction implemented according to aspects described herein. Thus, an average objective function value is obtained by averaging converged (or otherwise terminal) objective function values for respective 3D reconstructions. Such values of average objective functions establish the validity of the 3D reconstruction approach based on optimization of sizes and positions of spheres within a defined volume, as is described herein.

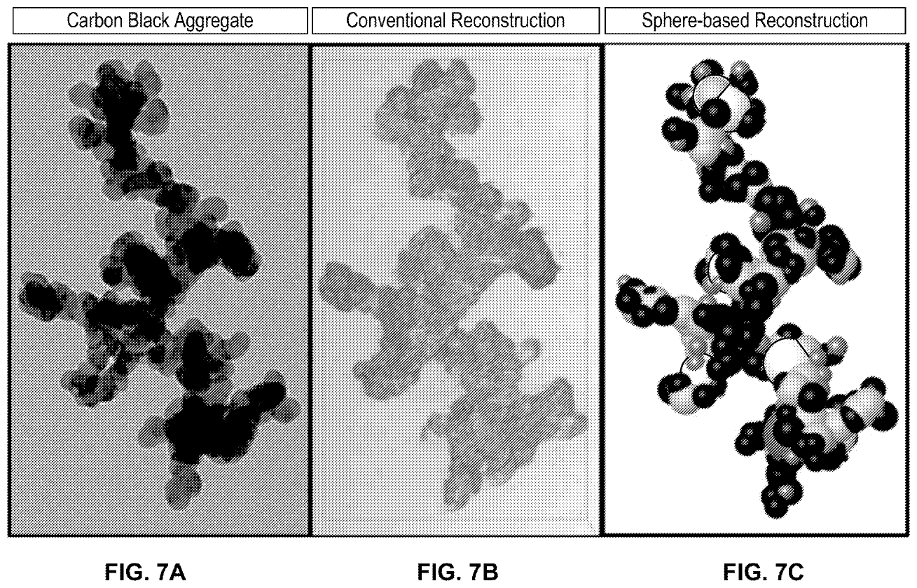

To further validate the 3D reconstruction approach of this disclosure, the reconstruction module 260 can reconstruct a carbon black aggregate that has been modeled using conventional TEM tomographic reconstruction. FIG. 7A is a no-tilt (0°) TEM micrograph of the carbon black aggregate. FIG. 7B is a no-tilt (0°) tomography view of the carbon black aggregate, as determined using conventional TEM tomographic reconstruction. Such a conventional reconstruction relies on a voxel-based approach and limited segmentation time. FIG. 7C is a non-tilt (0°) sphere-based 3D reconstruction view of the carbon aggregate, in accordance with aspects of this disclosure. Tone of grey shading of sphere indicates diameter range, as is noted hereinbefore in connection with FIG. 6. It is readily apparent from FIG. 7B that the conventional voxel-based reconstructions fails to achieve good surface definition in limited segmentation times. Good agreement is seen in macroscopic properties between the traditional tomography reconstruction and the sphere-based 3D reconstruction. Here, good agreement in macroscopic properties refers to good agreement in spatial extent of aggregate in a first in-plane direction (e.g., x direction) and a second in-plane direction (e.g., y direction) on the imaging plane, and also in curvature of aggregate boundary (where continuous, in the case of the rough, “voxelated” voxel-based reconstruction).

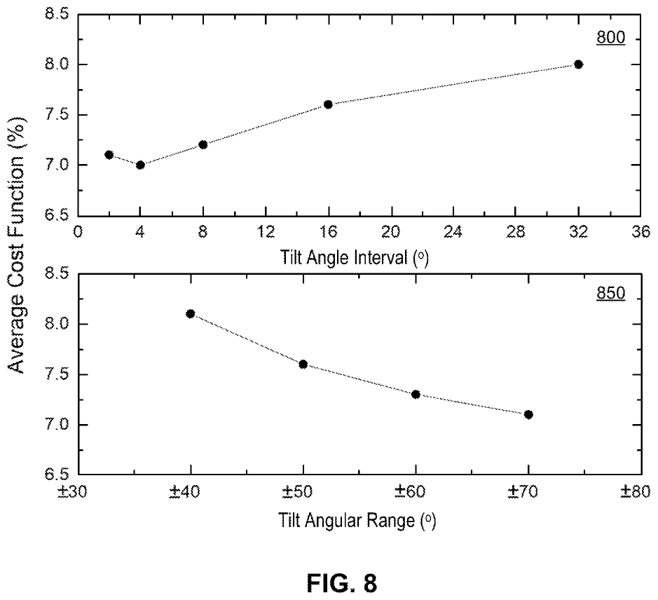

As is described herein, a series of TEM tilt images acquired through a tilt angular range [−Θ, Θ] at particular Δθ intervals can be used in the 3D reconstruction of a carbon black. Thus, the reconstruction module 260 can use various series of TEM tilt images according to different tilt angle intervals and tilt angular ranges in order to reconstruct the thirty N330 carbon black aggregates marked in FIG. 3. As mentioned, the 3D reconstruction process is iterative and, in some cases, converges and yields model data defining a 3D aggregate model. Upon convergence, a converged value of the objective function involved in the 3D reconstruction process also is determined. FIG. 8 presents a scatter plot 800 of converged objective function values, each averaged over the 30 N330 carbon black aggregates, using series of TEM tilt images acquired at five tilt angle intervals (2°, 4°, 8°, 16°, and 32°), at a tilt angular range [−70°,70°]. FIG. 8 also presents a scatter plot 850 of converged objective function values, each averaged over the thirty N330 carbon black aggregates, using series of TEM tilt images acquired at four tilt angular ranges ([−40°,40°], [−50°, 50°][−60°,60°], and [−70°,70°]), at a tilt angle interval Δθ=2°. The average objective function results indicate that 3D reconstruction with TEM tilt images acquired with tilt angle interval Δθ=8° performs nearly as well as the 3D reconstruction using TEM tilt images acquired tilt angle interval Δθ=2°. Thus, with one-fourth the number of images to acquire, the Δθ=8° series can yield a substantial reduction of data acquisition time relative to the Δθ=2° series. The reduction of the number of tilt TEM images that is achieved by reducing the tilt angular range does not yield comparable performance. Accordingly, image acquisition over tilt angular range [−70°,70°] may be desirable. Based on the results shown in FIG. 8, 3D reconstruction can be based on series of TEM tilt images acquired using tilt angular range [−70°,70°] range and Δθ=10°.

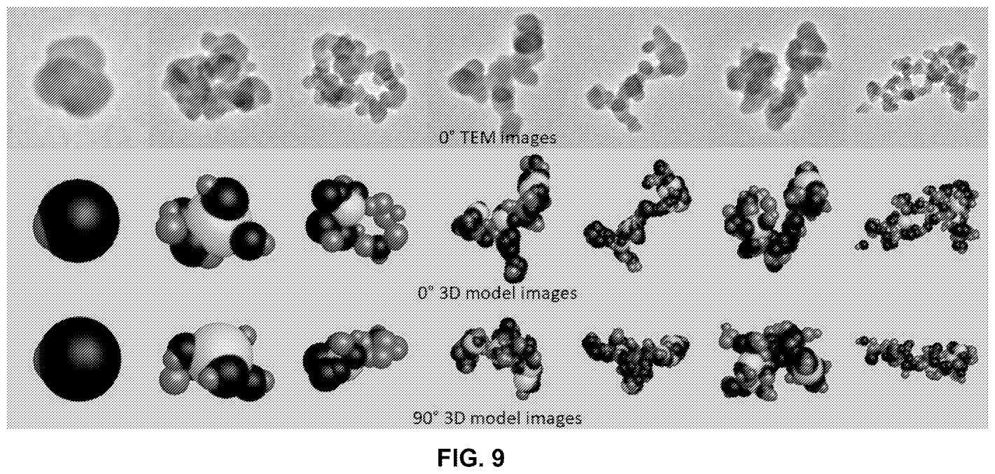

FIG. 9 illustrates a sample of seven reconstructed carbon black aggregates from the select 30 N330 carbon black aggregates marked in FIG. 3. Again, tone of grey shading of sphere indicates diameter range. Structural complexity of the seven aggregates increases across FIG. 9 from left to right (in reading orientation). Good agreement between 3D model projections and 2D images in terms of good match in pixel coverage between 0° TEM images and 0° 3D model projections. Additionally, it is observed that the aggregates having lower structure appear to have somewhat too few particles to properly model the carbon black aggregate volume and surface, and the higher structure aggregates have adequate numbers of particles. Without intending to be bound by interpretation or conjecture, such an observation can arise from using an estimator of the number of particles that is based on existing 2D analysis, as is described herein.

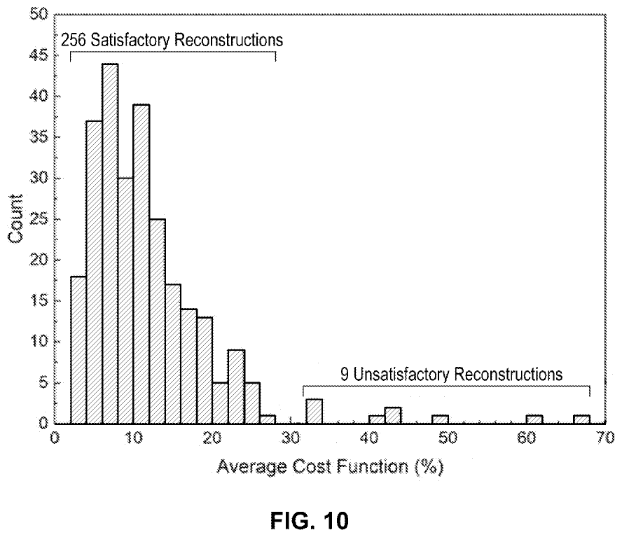

The 3D reconstruction approach of this disclosure can be implemented for multiple separate series of TEM tilt images of carbon blacks (e.g., N330 carbon blacks), for many individual carbon black aggregates depicted in those series. A first series of the multiple separate series is the series that includes the no-tilt TEM micrograph shown in FIG. 3. In one example scenario, the reconstruction module 260 can apply the 3D reconstruction process of this disclosure to a total of 265 potentially reconstructable aggregates. Here, a potentially reconstructable aggregate refers to a carbon black aggregate that is visible in all projections across a series of TEM tilt images, and for which carbon black aggregate the segmentation module 256 can determine an aggregate binary with satisfactory match of the template in each image in the series of TEM tilt images. Again, the 3D reconstruction process is iterative and, in some cases, converges and yields a converged value of the objective function involved in the 3D reconstruction process. A resulting distribution of converged average objective functions is shown in FIG. 10. By defining quality threshold value (e.g., 25%, 26%, 27%, or 30%), reconstructions can be determined to be satisfactory or unsatisfactory. A satisfactory reconstruction has a converged average objective function that is equal to less than then quality threshold value, whereas an unsatisfactory reconstruction has a converged average objective function that exceeds the quality threshold value. As is shown in FIG. 10, 256 reconstructions are satisfactory reconstructions. Each one of the satisfactory reconstructions is deemed of sufficient quality to contribute to a statistical analysis, and has final average objective function valued at less than 26% and contributes to an ensemble converged average objective function valued at 10.8%.

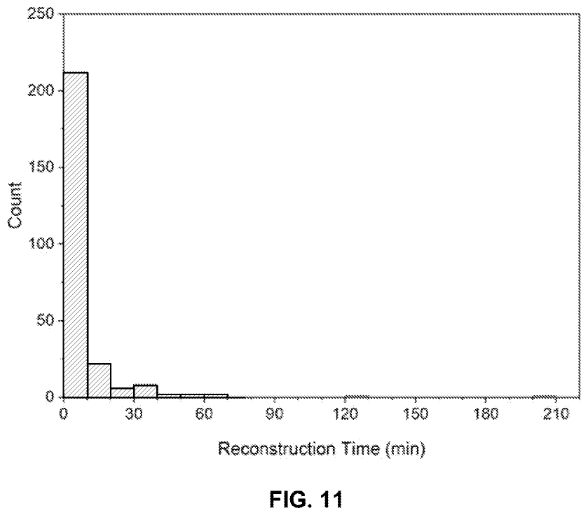

The computing device 250 (FIG. 2) can include one or more processors (not depicted in FIG. 2) that can be assembled in a single core or multiple cores. As an illustration of computational efficiency of the 3D reconstruction approach of this disclosure, the computing device 250 can perform 3D reconstruction of the 256 carbon black aggregates that have yielded satisfactory reconstructions in 48 hours of single-core processor compute time. The 3D reconstructions of relatively few, large carbon black aggregates consumed the bulk of the compute time. FIG. 11 illustrates a distribution of 3D reconstruction compute time.

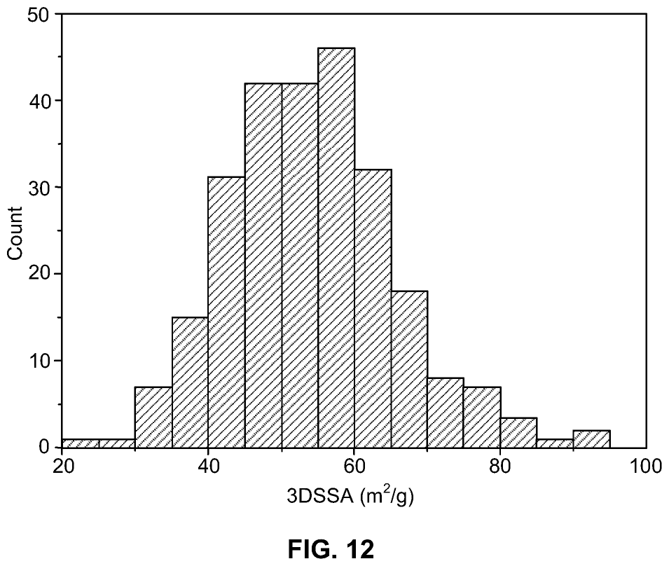

For each satisfactory reconstruction, the computing device 250 can include an analysis module 270 than can determine, using the determined 3D aggregate model, a 3DSSA for the carbon black aggregate corresponding to the satisfactory reconstruction. Here, for purposes of illustration, 3D specific surface area (3DSSA) a carbon black aggregate refers to total surface per unit of mass, where the total surface accounts for surfaces interfacing voids within the carbon black aggregate. A 3DSSA distribution for the 256 satisfactory reconstruction is shown in FIG. 12. The 3DSSA distribution yields a volume-weighted 3DSSA of 50.0 m²/g.

Post-reconstruction analysis is not limited to determining 3DSSA for a reconstructed carbon black aggregate. Indeed, after 3D aggregate reconstruction has been implemented, a general analysis of the morphology of a reconstructed carbon black aggregate can be implemented in a post-reconstruction analysis stage 140 of the process flow 100 (FIG. 1). More specifically, in the post-reconstruction analysis stage 140, various morphological properties of a reconstructed carbon black aggregate can be determined using a 3D aggregate model determined in the 3D reconstruction of that carbon black aggregate. As mentioned, the 3D aggregate model includes three-dimensional vector positions and sizes (e.g., diameters or radii) of each constituent sphere in the 3D aggregate model.

The modeling system 245, via the computing device 250 (FIG. 2), also can implement the post-reconstruction analysis stage 140. To that point, the analysis module 270 can determine the various structural properties of the reconstructed carbon black aggregate can be determined using the 3D aggregate model. Examples of the structural properties include 3DSSA (as described above), aggregate volume, aggregate convex hull volume, void volume, 3DVV, aggregate extent in X (Xₑₓₜₑₙₜ), aggregate extent in Y (Yₑₓₜₑₙₜ), and aggregate extent in Z (Zₑₓₜₑₙₜ), and “relative Z” (Zᵣₑₗ) Here, Zᵣₑₗ is an anisometry metric defined as the z dimension of the reconstructed carbon black aggregate relative to average of the x dimension and y dimension of the aggregate; that is, Zᵣₑₗ=2Zₑₓₜₑₙₜ/(Xₑₓₜₑₙₜ+Yₑₓₜₑₙₜ)

The analysis module 270 can determine mass and specific surface area distributions of overlapping sphere volumes of the reconstructed carbon black aggregate via 3D gridding (e.g., voxelization). The analysis module 270 can determine the 3DSSA of overlapping spheres by identifying and counting aggregate surface voxels, and correcting for the projected surface area coverage of voxels at different angles (correction factor can be averaged for spheres of varying voxel radii). More specifically, the analysis module 270 can apply a conversion (or correction) factor γ to a number of aggregate surface voxels in order to determine surface area of overlapping spheres. That surface area can then be used in the determination of 3DSSA. The conversion factor γ represents an empirical relationship between number of surface voxels and surface area for spherical particles. As such, γ can be determined empirically be converting surface voxels to surface area of spheres having a range of diameters spanning an interval that contains a mean diameter of particles in a type of carbon black aggregates under analysis (e.g., N330 carbon black aggregates). A conversion factor γ=1.30 can be obtained in such a fashion and is used in this disclosure. The analysis module 270 can determine occluded volume by determining an approximation to the aggregate convex hull. An analytic expression for the aggregate convex hull is unavailable for volumes bounded by discrete spheres. Accordingly, without intending to be bound by modeling, the convex hull can be determined by removing mass outside of bounding planes in rotational orientations varying by a defined angle increment (e.g., 6°) about all axes in a Cartesian coordinate system; summing the remaining volume as the convex hull volume; and subtracting the aggregate volume in order to arrive at the convex-hull—bounded intra-aggregate void volume. The intra-aggregate void volume divided by the aggregate volume defines the 3DVV.

Simply as an illustration, the analysis module 270 can determine aggregate volume, void volume, and 3DSSA in accordance with aspects described herein for 3D reconstructions of the 30 N330 carbon black aggregated marked in FIG. 3. The reconstruction module 260 generated the 3D reconstructions using TEM tilt images acquired using [±70°,70°] and Δθ=10°. The analysis module 270 determined dimensional extents of the aggregates in X, Y, and Z. Data for the 30 reconstructed aggregates are shown in Table 1. In that table, “Agg. ID” refers to the number adjacent to the marked aggregates in FIG. 3.

TABLE 1
Morphological properties of 30 N330 carbon black
aggregates from sphere-based 3D reconstructions

| Agg. ID | Spheres | Xₑₓₜₑₙₜ | Yₑₓₜₑₙₜ | Zₑₓₜₑₙₜ | Zᵣₑₗ | Agg Vol. | 3DVV | 3DSSA |
|---|---|---|---|---|---|---|---|---|
| — | — | (nm) | (nm) | (nm) | — | (nm³) | — | (m²/g) |
| 7 | 118 | 172 | 225 | 130 | 0.655 | 653000 | 1.52 | 61.2 |
| 8 | 99 | 207 | 215 | 142 | 0.672 | 1093000 | 1.32 | 56.4 |
| 10 | 11 | 189 | 155 | 135 | 0.786 | 1259000 | 0.34 | 32.3 |
| 16 | 2 | 84 | 80 | 80 | 0.980 | 256000 | 0.00 | 41.7 |
| 18 | 12 | 78 | 100 | 62 | 0.692 | 126000 | 0.71 | 78.0 |
| 20 | 7 | 82 | 119 | 73 | 0.733 | 204000 | 0.33 | 57.0 |
| 21 | 70 | 204 | 217 | 175 | 0.833 | 1186000 | 1.22 | 50.2 |
| 22 | 13 | 97 | 132 | 73 | 0.642 | 238000 | 0.46 | 57.3 |
| 23 | 5 | 77 | 94 | 68 | 0.804 | 180000 | 0.24 | 58.9 |
| 26 | 31 | 140 | 120 | 58 | 0.449 | 256000 | 0.69 | 64.9 |
| 28 | 5 | 100 | 67 | 60 | 0.720 | 131000 | 0.24 | 61.9 |
| 29 | 18 | 251 | 222 | 150 | 0.636 | 2052000 | 0.73 | 36.7 |
| 30 | 145 | 436 | 277 | 160 | 0.450 | 3212000 | 2.15 | 56.1 |
| 31 | 5 | 132 | 90 | 82 | 0.737 | 382000 | 0.11 | 42.1 |
| 32 | 30 | 135 | 195 | 132 | 0.798 | 781000 | 0.89 | 50.0 |
| 33 | 12 | 80 | 100 | 67 | 0.741 | 112000 | 0.73 | 81.9 |
| 34 | 26 | 244 | 150 | 122 | 0.619 | 1120000 | 0.66 | 42.4 |
| 36 | 9 | 129 | 117 | 73 | 0.599 | 258000 | 0.51 | 59.8 |
| 37 | 6 | 100 | 105 | 85 | 0.829 | 279000 | 0.30 | 51.8 |
| 38 | 59 | 204 | 129 | 117 | 0.704 | 555000 | 1.25 | 61.3 |
| 39 | 103 | 185 | 297 | 187 | 0.775 | 1215000 | 2.14 | 64.4 |
| 40 | 4 | 95 | 85 | 68 | 0.759 | 171000 | 0.33 | 65.5 |
| 41 | 76 | 154 | 195 | 107 | 0.612 | 601000 | 1.04 | 56.2 |
| 42 | 62 | 164 | 232 | 130 | 0.658 | 1037000 | 1.17 | 50.7 |
| 43 | 149 | 245 | 256 | 150 | 0.600 | 790000 | 2.30 | 60.0 |
| 44 | 4 | 75 | 70 | 48 | 0.667 | 85000 | 0.21 | 73.6 |
| 46 | 90 | 155 | 324 | 139 | 0.578 | 1090000 | 1.47 | 55.7 |
| 47 | 91 | 145 | 152 | 109 | 0.730 | 479000 | 1.65 | 65.4 |
| 49 | 188 | 319 | 451 | 202 | 0.525 | 2363000 | 2.81 | 56.3 |
| 53 | 5 | 107 | 110 | 72 | 0.662 | 281000 | 0.22 | 50.2 |

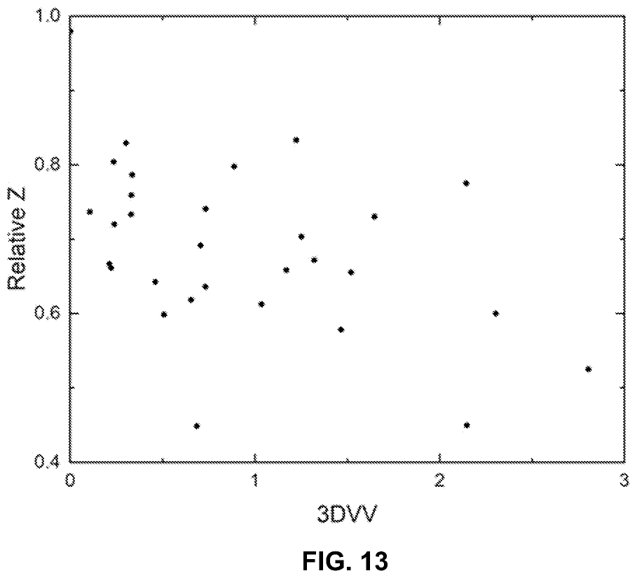

According to existing analysis of carbon black aggregates that is available in existing literature related to carbon black, the degree of anisometry or carbon black aggregates increases with aggregate structure. FIG. 13 illustrates Zᵣₑₗ for the 30 N330 carbon black aggregates identified in Table I and marked in FIG. 3. It is observed that all aggregates have a lesser extent in Z than in X and Y. There is also some dependence of anisometry on aggregate structure measured via 3DVV, with more highly structured aggregates tending to have more anisometry (that is, lower Zᵣₑₗ values). Accordingly, the results obtained with the 3D reconstruction approach of this disclosure and shown in FIG. 13 support existing findings and provide a direct measure of aggregate anisometry. That is, anisometry can be characterized by Zᵣₑₗ, which metric can be determined directly from a 3D reconstruction in accordance with aspects of this disclosure. Such a direct measure is unavailable in existing analyses of carbon black aggregates because the existing analyses of carbon black aggregates are based on 2D data and merely infer anisometry in the third dimension (e.g., z direction).

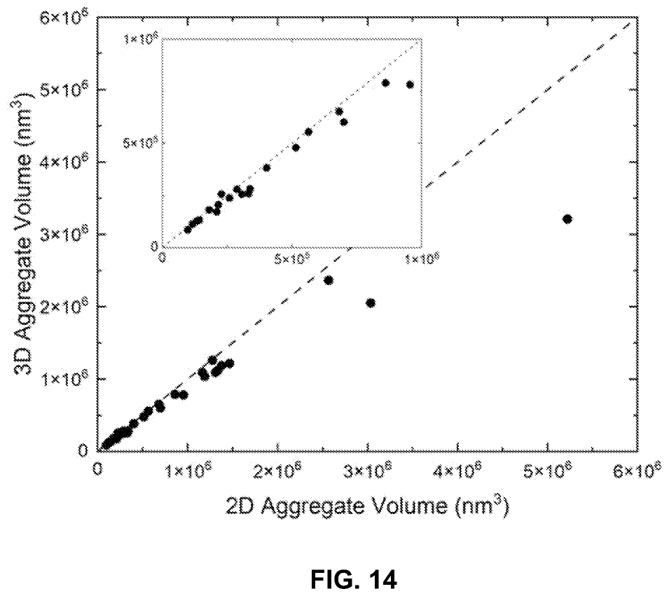

For 3D aggregate models according to aspects of this disclosure, aggregate volume (denoted “Agg. Vol” in Table 1) can be calculated directly. As is shown in FIG. 14, particularly in the inset thereof, the 3D aggregate volume is correlated with 2D-based volume for lower volume (and generally lower structure) aggregates, with the 2D-based volume increasing relative to the 3D-based volume as the volumes increase. Without intending to be bound by theory or modeling, this can be understood by considering the ramifications of anisometry on the 2D-based volume measurements. Specifically, existing 2D estimator considers aggregate isometry, so that reduction in the aggregate extent in the Z-direction relative to the case for isometric aggregates renders the 2D-based calculation larger than the directly determined 3D-based volume according to the 3D reconstruction of this disclosure.

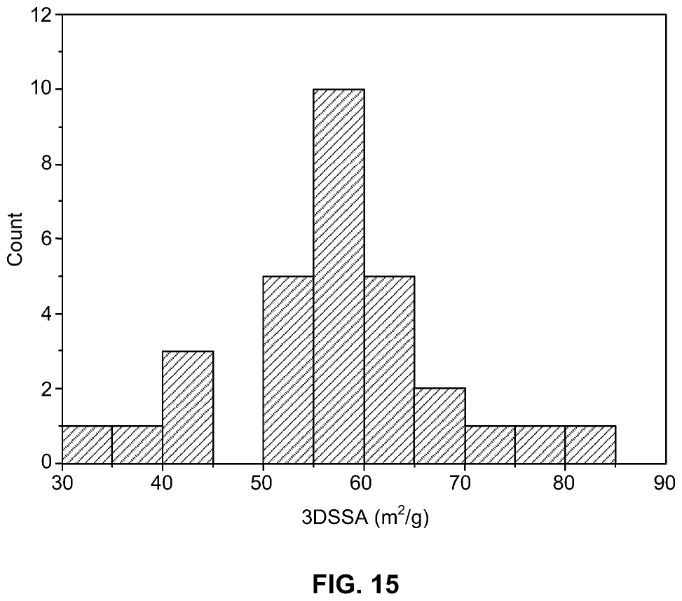

In cases where density is known, 3DSSA can be determined from voxelization of the 3D aggregate models (as is described herein) and can be related directly to bulk colloidal measurements. The individual aggregate 3DSSA values shown in Table 1 range from 30 m²/g to 85 m²/g. As is illustrated in FIG. 15, the distribution of 3DSSA values for 30 reconstructed N330 carbon black aggregates is approximately normal or log-normal. The volume-weighted average of 3DSSA is 52.5 m²/g. This value is less than the typical statistical thickness surface area (STSA) value for N330 carbon black (75 m²/g) by about 30%. Without intending to be bound by interpretation, such a difference may originate from interior regions of a reconstructed carbon black aggregate having rather excessive mass and spherical particles of rather large diameters. Such factors, individually and in combination, decrease the modeled specific surface area (SSA). It is noted that the analysis module 270 can determine SSA by counting boundary pixels just inside a carbon aggregate. Counting space boundary voxels just outside the carbon aggregate instead increases the 3DSSA value to about 55 m²/g, a value that still is less than the typical STSA value.

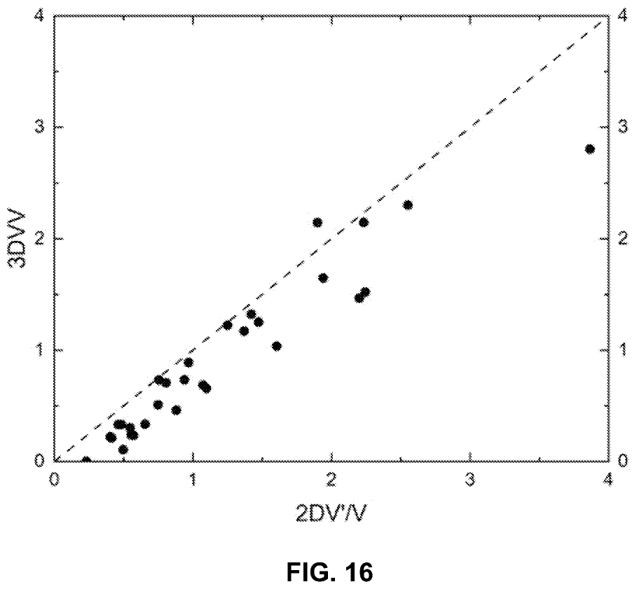

The existing 2D V′/V supports an expected fundamental relation between OAN and relative void volume. Such a relationship is the quotient of aggregate void volume (denoted by V′) divided by aggregate volume (denoted by V). For this relation, the generally accepted approximation 1.9 g/cm³ for carbon black density can be used to convert volume to mass, and the adjustment to OAN units (cm³/100 g) results in a theoretical slope of OAN to true relative void volume of 100/1.9≈53. The 2D V′/V can be referred to aggregate absorptivity factor or structure factor, and can represent relative void volume. Such a relationship is derived from 2D-based approximations of the diameter of the space occupied by the aggregate. Without intending to be bound by conjecture, if 3DVV were a useful measure of relative void volume, 3DVV would be expected to follow a similar relationship. To that point, FIG. 16 indeed presents a linear relationship for the 30 aggregates measured in this disclosure. The slope of that linear relationship is near unity and there is a small offset. The 3DVV values are about 0.3 units lower than the 2D V′/V values (calculated from single, 0° aggregate projections). The weighted void volume determined via the convex hull approximation was 1.39, 18% less than the 2D V′/V relative void volume of 1.69 for these 30 N330 carbon black aggregates.

Without intending to be bound by interpretation or conjecture, the combination of 25% to 30% lower specific surface area with 18% lower 3DVV relative to expectations from 2D analysis could be attributable to the inclusion of excess mass near the center of the 3D aggregate models. The filling of small spaces that are not visible in the binaries due to overlap of adjoining aggregate mass might slightly increase mass and decrease surface area. The combined effect of increasing mass and decreasing surface area could decrease the determined 3DSSA and 3DVV.

Having access to morphological properties of a carbon black can permit analyzing how those properties affect the interaction of the carbon black with rubber via occlusion. Occluded volume may be approximated as the volume contained within a convex hull surrounding a carbon black aggregate. Existing literature related to carbon black includes relations directly tying carbon black occluded volume measurements to oil absorption number (which number also has been referred to as DBP). As mentioned, OAN is a bulk measure of amount of oil necessary to just fill intra-aggregate voids associated with a carbon black aggregate. That existing literature also raised issues with the concept of occluded rubber “within” carbon black aggregates (and shielded from strain to some geometrical extent), since each intra-aggregate rubber volume element might actually experience a degree of occlusion measured on a continuum, depending on the stress shielding at its location.

The issue of what volume is “within” an aggregate can be addressed with data indicative of the 3D mass distribution of carbon black aggregates. Such a distribution can be modeled based on spatial distribution of spheres forming a 3D aggregate model in accordance with this disclosure. The analysis module 270 (FIG. 2) using such data, can implement nano-scale studies involving finite element analysis (FEA) of rubber deformation in proximity to 3D aggregate models of carbon black aggregates in accordance with this disclosure. Analysis of many other applications also may be implemented using such data. Because carbon black aggregates break down to some degree in rubber compounding, carbon blacks isolated from rubber compounds also can be analyzed. Accordingly data defining 3D aggregate models as is described herein provide a rich base of 3D carbon black aggregate morphological data that can be readily applied to modelling reinforcement in rubber and also to guiding product development efforts.

In addition to occlusion and reinforcement, many other rubber properties substantively depend on carbon black morphology. Again, having access to morphology data indicative of morphological properties of carbon black aggregates and model data representative of 3D carbon black mass distribution can elucidate aspects of properties such as permeability, conductivity, and hysteresis, and also can permit predicting and/or engineering the impact of one or more of those properties. Simply as an illustration, existing literature related to carbon black has probed the impact of filler morphology on permeability via simulated aggregate modeling and diffusion. Data indicative of 3D carbon black morphology may validate the diffusion model in that existing literature and also may elucidate the geometrical optimization of carbonaceous solid barriers to gas permeation. Data indicative of aggregate mass distributions in 3D, especially at carbon black aggregate peripheries, can permit establishing filler-filler networking characteristics of polymers. Such networking characteristics can affect static properties, such as conductivity, and also affect hysteretic transient filler networking (known as the Payne effect). The wealth of morphology data afforded by the 3D reconstruction approach of this disclosure is unavailable from existing tomographic reconstruction technologies. Therefore, embodiments of this disclosure can provide a superior data framework applicable to the understanding and engineering of such phenomena. Further, as is described herein, such a superior data framework can be used to identify type of carbon black of carbon black aggregates based on machine-learned predictive models. Such models can be trained using the wealth of data supplied by the data framework. Accordingly, embodiments of this disclosure can provide various practical analytic tools for the study of carbon black aggregates.

With further reference to FIG. 2, the computing device 250 also can include a reporting module 274 that can supply 3D aggregate models and/or morphology data of carbon black aggregates. Such information can be supplied in numerous ways. In some embodiments, to supply 3D aggregate models, the reporting module 274 can retain the 3D aggregate models in one or more memory devices (e.g., the memory 278), and can configure an API to access the 3D aggregate models. In addition, or in some cases, the reporting module 274 can supply a 3D aggregate model by causing presentation of a visualization of the 3D aggregate model at a user interface. The reporting module 274 can cause a display device 280 to present such a user interface. As is illustrated in FIG. 2, the display device 280 can be functionally coupled to the computing device 250. Such a coupling can be provided by a wireline interface and/or a wireless interface (such interfaces represented by a segment having open arrows). In other cases, the display device 280 can be integrated into the computing device 250. Similarly, to supply the morphology data, e.g., values of a morphological property of the carbon aggregate, the reporting module 274 can retain the values in one or more memory devices (e.g., the memory 278), and can configure an API to access one or multiple values of a particular morphological property. In addition, or in other embodiments, to supply such values, the reporting module 274 can cause presentation, at a user interface, of visual elements indicative of one or multiple values of the one or more morphological properties. Again, that user interface can be presented at the display device 280. Further, or in yet other embodiments, to supply such values, the reporting module 274 can cause transmission of an electronic message (an email, for example) conveying one or multiple values of the one or more morphological properties. The reporting module 274 also can cause presentation of a notification that a 3D aggregate model is available for analysis, including visualization of the 3D aggregate model.

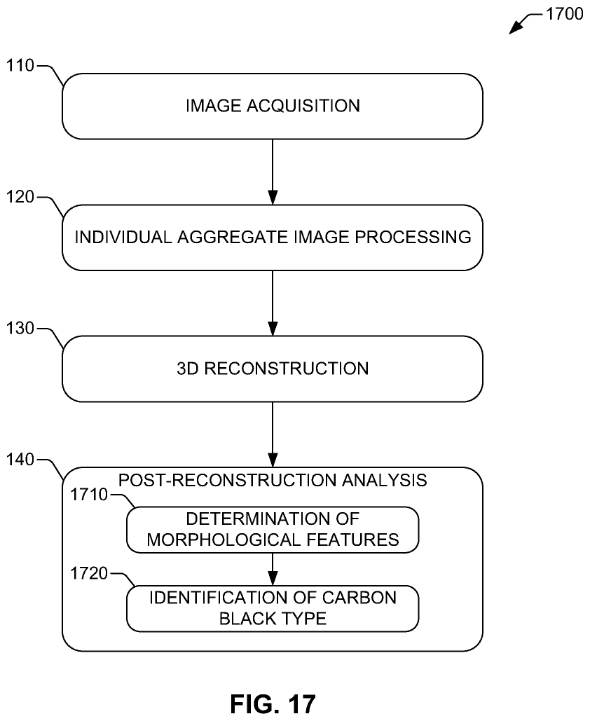

The 3D reconstruction of a carbon black aggregate and post-reconstruction analysis of the carbon black aggregate can be used to identify a type of carbon black of the carbon black aggregate. To that end, as is illustrated in FIG. 17, the post-reconstruction analysis 140 can be expanded to include sub-stages that can permit identifying, based on values of morphological properties of the carbon black aggregate, the type of carbon black corresponding to the carbon black aggregate. At a first sub-stage 1710, morphological features can be determined. A morphological feature can be a value of a morphological property P of the carbon black aggregate. Accordingly, a feature vector f can be determined at the first sub-stage 1710, where the feature vector f can have M components corresponding to properties P₁, P₂, . . . P_{M}, and each component can have a value determined using the 3D aggregate model of the carbon black aggregate determined at stage 130, as is described herein. In some cases, M=7 and the morphological properties can include Xₑₓₜₑₙₜ, Yₑₓₜₑₙₜ, Zₑₓₜₑₙₜ, Zᵣₑₗ, aggregate volume, 3DVV, and 3DSSA. The disclosure is not limited to that number of morphological properties, and fewer or more than seven morphological properties can be contemplated. A second sub-stage 1720 can identify a type of carbon black by applying a prediction model to the feature vector f The prediction model can be a machine-learned model configured (e.g., trained) to designate a carbon black aggregate as being of a particular type of carbon black. For example, the prediction model can be a classification model configured (e.g., trained) to solve a multi-task classification problem.

In order to generate such a prediction model, the operating environment 200 (FIG. 2) can be used to generate datasets corresponding to respective carbon black aggregates pertaining to known types of carbon black (e.g., N110 carbon black, N220 carbon black, N330 carbon black, N550 carbon black, N660 carbon black, N772 carbon black, N880 carbon black, N990 carbon black, and the like). Each dataset of the datasets can be labeled according to a type of carbon black and can include an instance of feature vector f=(P₁, P₂, . . . P_{M}). Such labeled datasets can be referred to as labeled data and can be retained in one or more memory devices (referred to as a labeled data repository). The prediction model can then be trained to solve a multi-task classification problem on a feature vector corresponding to multiple values of morphological properties of a carbon black aggregate.

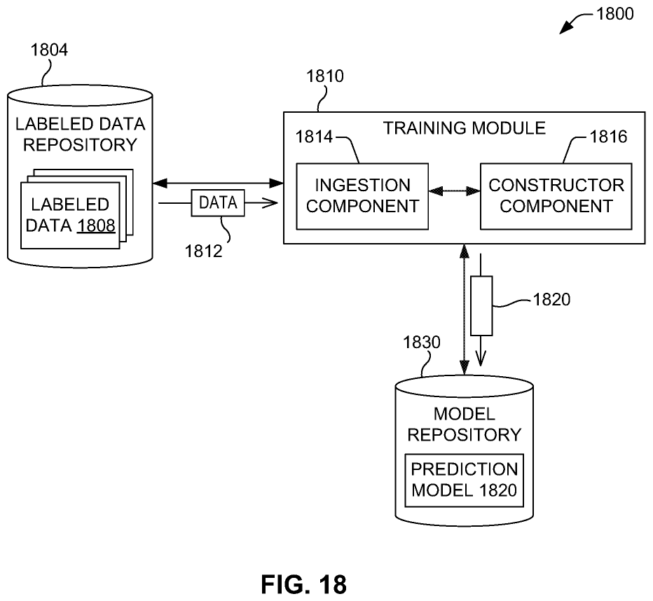

FIG. 18 illustrates an example system 1800 to generate a prediction model configured to designate a carbon black aggregate as being of a particular type of carbon black, in accordance with one or more embodiments described herein. As mentioned, the prediction model can be embodied in a classification model configured to solve a multi-task classification problem. The system 1800 can include a training module 1810. The training module 1810 can implement supervised, unsupervised, and/or semi-supervised (e.g., reinforcement-based) machine-learning models. The training module 1810, via an ingestion component 1814, for example, can obtain different types of training data. Because the prediction model can be trained to solve a multi-task classification problem on a feature vector corresponding to multiple values of morphological properties of a carbon black aggregate, the ingestion component 1814 can obtain labeled data 1812 from one or more memory devices 1804 (referred to as labeled data repository 1804). The labeled data 1812 can define multiple labeled feature vectors from a group of several labeled features vectors defined by labeled data 1808. The feature vectors may have been labeled as part of 3D reconstruction of carbon black aggregates of a carbon black of a known type (e.g., N110 carbon black, N220 carbon black, N330 carbon black, N550 carbon black, N660 carbon black, N772 carbon black, N880 carbon black, N990 carbon black, or similar), for example. A label of a labeled feature vector can include a textual element. Each label for a labeled feature vector designates a type of carbon black—e.g., N110 carbon black, N220 carbon black, N330 carbon black, N550 carbon black, N660 carbon black, N772 carbon black, N880 carbon black, N990 carbon black, or similar.

The training module 1810 also includes a constructor component 1816 that can operate on the data 1812 obtained by the ingestion component 1814. By operating on the data 1812, the constructor component 1816 can train the detection model using at least a subset of the labeled images 1808. As mentioned, the prediction model can be a classification model that can be trained to solve a multi-task classification problem and designate a carbon black aggregate as pertaining to one of multiple categories, each category representing a type of carbon black. To train the prediction model, the constructor component 1816 can determine, using the data 1812, a solution to an optimization problem with respect to a cost function. The solution can be determined iteratively, and at each iteration the cost function yields a value based on evaluation of differences between known labels for respective carbon black aggregates and predicted labels for the respective carbon black aggregates. The predicted labels being predicted or otherwise determined by a current iteration of the classification model; that is, the classification model as defined at that current iteration. After a solution has been determined, the solution results in model parameters that minimize the cost function. The model parameters define a trained prediction model 1820. The training module 1810, via the constructor component 1816, for example, can retain the trained prediction model 1820 in one or more memory devices 1830 (referred to as model repository 1830).

In some embodiments, the training module 1810 can be hosted by the computing device 250, the labeled data repository 1804 and the model repository 1830 can be part of the memory 278. In other embodiments, another computing device or system of computing devices functionally coupled to the computing device 250 can include the training module 1810, and the labeled data repository 1804 and the model repository 1830.

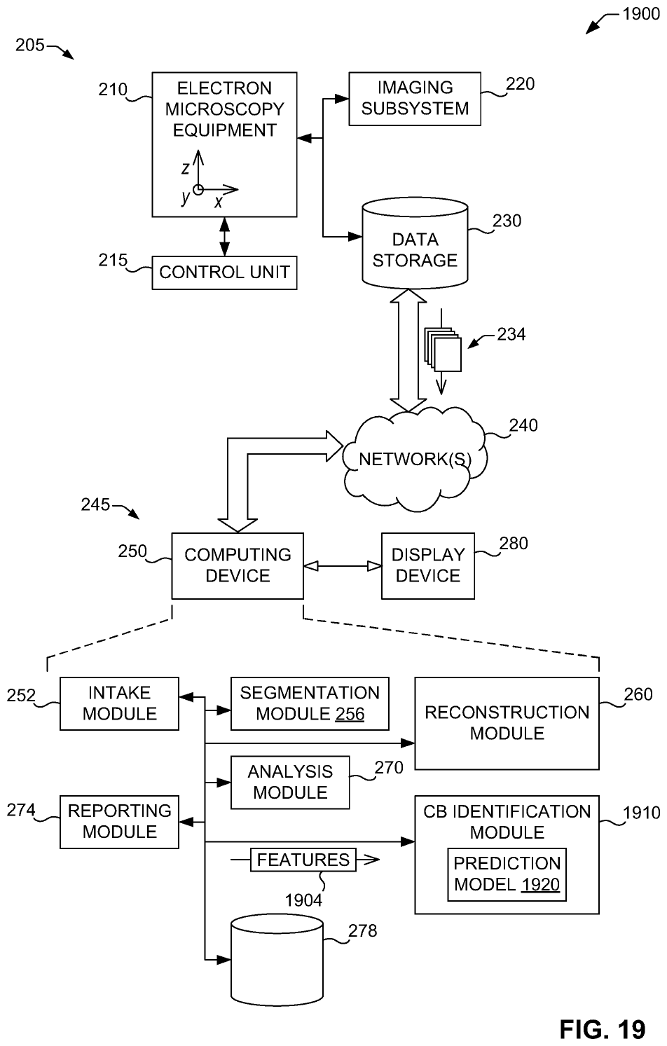

A trained prediction model (e.g., prediction model 1820) can be used to identify type of carbon black to which a carbon black aggregate pertains. To that end, the carbon black aggregate can be probed according using electron microscopy and modeled in accordance with aspects described herein in connection with the process flow 1700 (FIG. 17). As such, the operating environment 1900 can be utilized to generate a 3D aggregate model for the carbon black aggregate by determining a 3D reconstruction of that carbon black aggregate. The 3D aggregate model can be generated by implementing, via the computing device 250 (FIG. 19), stage 110 to stage 130 in the process flow 1700. As is described herein, the analysis module 270 can generate, based on the 3D aggregate model, values of respective morphological properties of the carbon black aggregate. The analysis module 270 can cast such values as a feature vector f, where each component off corresponds to a morphological property P of the carbon black aggregate and has a value that is equal to the value determined by the analysis module 270 for the morphological property P. As is described herein, f can have M components corresponding to properties P₁, P₂, . . . _{PM}, where such properties can be the same as the morphological properties used to generate a training dataset for a prediction model 1920. In FIG. 19, an M-tuple defining an instance of the feature vector f is denoted as features 1904. That is, a first carbon black aggregate that is probed, modeled, and analyzed according to the process flow 1700 has first features 1904, e.g., an instance of f=(P₁, P₂, . . . P_{M}); and a second carbon black aggregate that is probed, modeled, and analyzed according to the process flow 1700 has another instance of f=(P₁, P₂, . . . P_{M}). As mentioned, in some cases, M=7 and the morphological properties can include Xₑₓₜₑₙₜ, Yₑₓₜₑₙₜ, Zₑₓₜₑₙₜ, Zᵣₑₗ, aggregate volume, 3DVV, and 3DSSA.

For a particular carbon black aggregate, the analysis module 270 can determine features 1904 and can pass the determined features 1904 to a carbon black (CB) identification module 1910. In some cases, the features 1904 can be passed by sending data indicative of the features 1904. In other cases, the features 1904 can be passed by reference—that is, the analysis module 270 can retain the features 1904 within the memory 278 (FIG. 19), and can then send, to the CB identification module 1910, a datum or data pointing to a memory block where the features 1904 are retained within the memory 278 (FIG. 19). The CB identification module 1910 can then readout or otherwise obtain the features 1904 form that memory block within the memory 278.

The CB identification module 1910 can apply the prediction model 1920 to the features 1904. In response to applying the prediction module 1920, the CB identification module 1910 can generate multiple numerical weights corresponding to respective types of carbon black. Such respective types and a number thereof can the same as the group of types and number thereof used to train the prediction model 1920. Based on the multiple numerical weights, the CB identification module 1910 can designate the carbon black aggregate associated with the features 1904 as being of a type of carbon black. For example, the type of carbon black can have the largest weight of the multiple numerical weights. The CB identification module 1910 can then cause the display device 280 to present a notification or indicia indicative of the type of carbon black.

In some embodiments, rather than analyzing a carbon black aggregate individually, the computing device 250 in FIG. 19 can analyze a group of carbon black aggregates in order to predict one or more types of carbon black representative of the group of carbon black aggregates. To that end, respective sets of features 1904 can be determined for carbon black aggregates that form the group of carbon black aggregates. For each one of sets, the CB identification module 1910 can determine respective predictions of a type of carbon black. The CB identification module 1910 can then evaluate the respective predictions and based on the evaluation, the CB identification module 1910 can determine one or more representative types of carbon black for the group of carbon black aggregates. The CB identification module 1910 can rank the one or more respective types, and can then cause the display device 280 to present a notification or indicia indicative of a ranking of the one or more respective types.

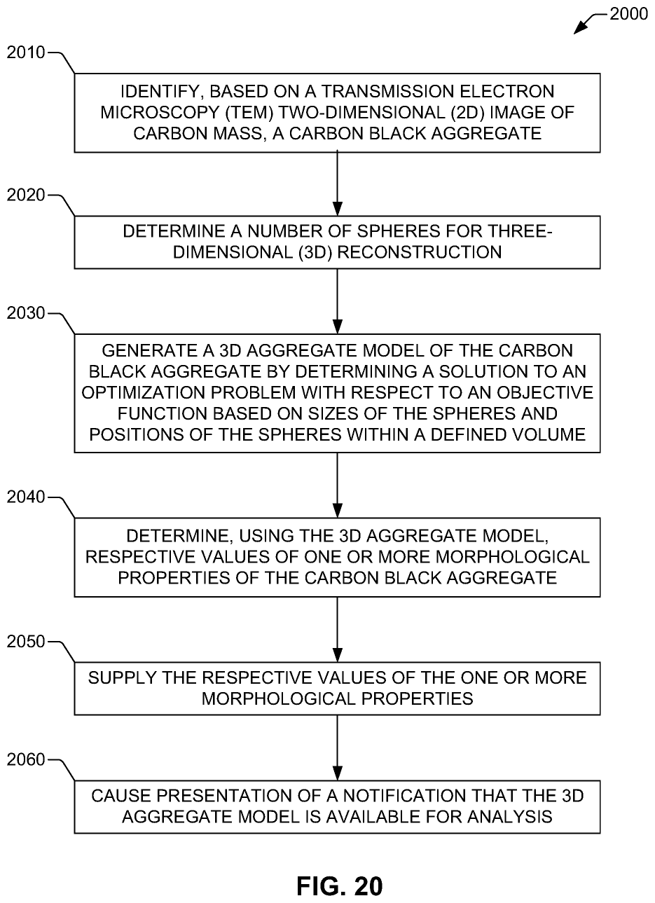

In view of the aspects described herein, example methods that may be implemented in accordance with this disclosure can be better appreciated with reference, for example, to the flowcharts in FIGS. 20-24. For the sake of simplicity of explanation, the example methods disclosed herein are presented and described as a series of blocks (with each block representing an action or an operation in a method, for example). However, the example methods are not limited by the order of blocks and associated actions or operations, as some blocks may occur in different orders and/or concurrently with other blocks from those that are shown and described herein. Further, not all illustrated blocks, and associated action(s), may be required to implement an example method in accordance with one or more aspects of the disclosure. Two or more of the example methods (and any other methods disclosed herein) may be implemented in combination with each other. It is noted that the example methods (and any other methods disclosed herein) may be alternatively represented as a series of interrelated states or events, such as in a state diagram.

FIG. 20 illustrate an example of a method 2000 for providing a 3D reconstruction and analysis of a carbon black aggregate, in accordance with one or more embodiments of this disclosure. A computing device or a system of computing devices can implement the example method 2000 in its entirety or in part. To that end, each one of the computing devices includes computing resources that may implement at least one of the blocks included in the example method 2000 and other methods described herein. The computing resources comprise, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. The computing device can embody the computing device 250 and, thus, can host at least one of the intake module 252, the segmentation module 256, the reconstruction module 260, the analysis module 270, and the reporting module 274. Accordingly, the computing device can implement the example method 2000 by executing one or more instances of such modules.

At block 2010, the computing device can identify, based on a TEM 2D image of a carbon mass, a carbon black aggregate. To that end, in some cases, the computing device can apply one or more machine-vision techniques including, for example, object-localization techniques such as the template matching described herein.

At block 2020, the computing device can determine a number of spheres N_{S} for 3D reconstruction of the carbon black aggregate. To that end, in accordance with aspects described herein, the computing device can determine a volume of the carbon black aggregate (V_{agg}) and particle volume (Vₚₐᵣₜ). The computing device can then determine Nₛ by determining the quotient of V_{agg} divided by Vₚₐᵣₜ. That is, N_{S}=V_{agg}/Vₚₐᵣₜ

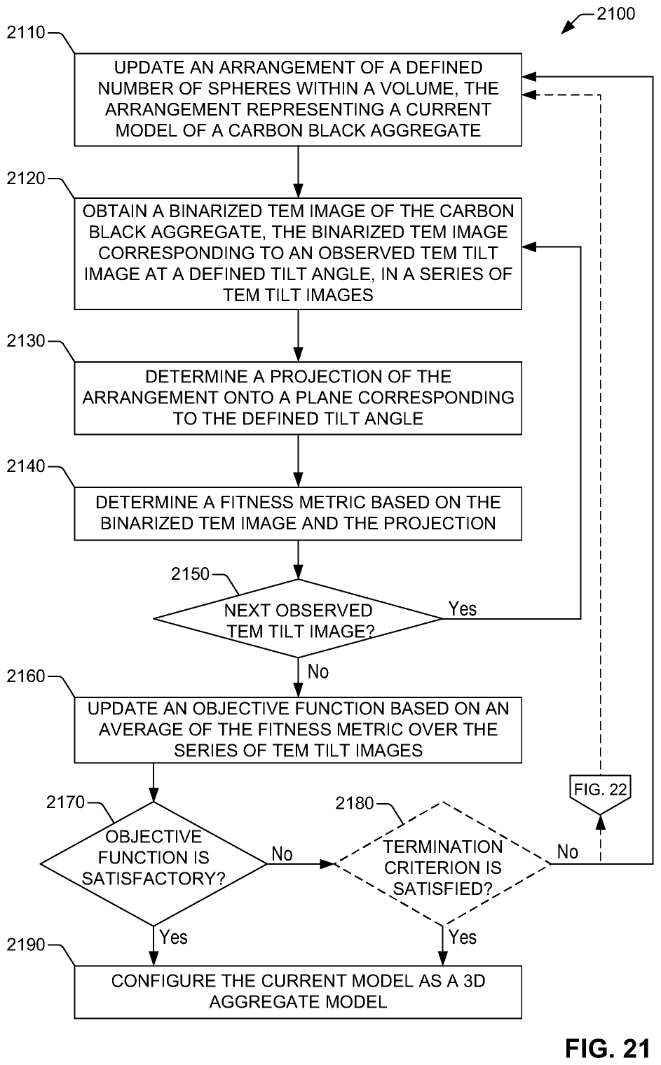

At block 2030, the computing device can generate a 3D aggregate model of the carbon black aggregate by determining a solution to an optimization problem with respect to an objective function based on sizes of the spheres and positions of the spheres within a defined volume. In some cases, to generate the 3D aggregate model in such a fashion, the computing device can implement the example method 2100 shown in FIG. 21.

At block 2040, the computing device can determine, using the 3D aggregate model, respective values of one or more morphological properties of the carbon black aggregate. The one or more morphological properties include at least one of aggregate anisometry metric (e.g., Zᵣₑₗ), aggregate volume, relative 3D void volume (3DVV), or 3D specific surface area (3DSSA).

At block 2050, the computing device can supply the respective values of the one or more morphological properties. In some embodiments, supplying such values can include retaining the values in one or more memory devices, and configuring an API for access to one or multiple values of a particular morphological property of the one or more morphological properties. In addition, or in other embodiments, supplying such values can include causing a display device to present visual elements indicative of one or multiple values of the one or more morphological properties. Further, or in yet other embodiments, supplying such values can include sending an electronic message (an email, for example) conveying one or multiple values of the one or more morphological properties.

At block 2060, the computing device can cause presentation of a notification that the 3D aggregate model is available for analysis. Analysis can include visualization of the 3D aggregate model at a user interface presented at a display device (e.g., display device 280). In some cases, the display device can present the notification at that user interface or another user interface.

FIG. 21 illustrate an example of a method 2100 for iteratively generating a 3D aggregate model of a carbon black aggregate, in accordance with one or more embodiments of this disclosure. A computing device or a system of computing devices can implement the example method 2100 in its entirety or in part. The computing device that implements the example method 2000 also can implement the example method 2100. Accordingly, the computing device can implement the example method 2100 by executing the reconstruction module 260.

At block 2110, the computing device can update an arrangement of a defined number of spheres within a defined volume. The defined number of spheres can be AT, determined at block 2020 of example method 2000 (FIG. 20). The arrangement is defined by a configuration of positions and a configuration of sizes of the number of spheres within the define volume. Thus, updating the arrangement can include updating the configuration of positions and updating the configuration of sizes in accordance with aspects described herein. For example, updating the arrangement can include modifying a position of a first sphere of the spheres and maintaining second positions of second spheres of the spheres. In addition, or in some cases (based on current iteration or reconstruction step) modifying a size of the first sphere and maintaining second sizes of the second spheres. In some embodiments, updating such an arrangement can include performing a simulated-annealing update, performing a genetic algorithm update, or performing a conjugate-gradient steepest descent update.

At block 2120, the computing device can obtain a binarized TEM image of the carbon black aggregate. The binarized TEM image corresponds to an observed TEM tilt image at a defined tilt angle, in a series of TEM tilt images. The observed TEM tilt image can be one of a series of TEM tilt images over a tilt angular range [−Θ,Θ], at consecutive tilt angle intervals Δθ. In one example, Θ=70° and Δθ=10°, and the defined tilt angle pertains to the set {−70°, −60°,−50°,−40°,−30°,−20°,−10°, 0°, 10°, 20°, 30°, 40°, 50°, 60°, 70°}.

At block 2130, the computing device can determine a projection of the arrangement of the defined number of spheres onto a plane corresponding to the defined tilt angle.

At block 2140, the computing device can determine a fitness metric based on the binarized TEM image and the projection. The fitness metric can be the number of mismatched pixels (or, in some cases, a function of that number) resulting from a pixel-wise difference between the binarized TEM image and the projection. Thus, determining the fitness metric can include determining that number of mismatched pixels.

At block 2150, the computing device can determine if another observed TEM tilt image is available for processing in the series of TEM tilt images. A affirmative determination (“Yes” branch) results in the flow of the example method 2100 being directed to block 2120, for another iteration of the implementation of block 2120, block 2130, block 2140, and block 2150. A negative determination (“No” branch) at block 2150 results in the flow of the example method 2100 being directed to block 2160, where the computing device can update an objective function based on an average of the fitness metric over the series of TEM tilt images.

At block 2170, the computing device can determine if a value of the objective function is satisfactory. To that end, the computing device can determine if the value of the objective function satisfies a convergence criterion. The convergence criterion can dictate that the magnitude of a difference between the value and a prior value in an immediate prior determination of the objective function is less than or equal to a threshold value. An affirmative determination (“Yes” branch) is indicative of convergence and results in the flow of the example method 2100 being directed to block 2190, where the computing system can configure the current model as a 3D aggregate model of the carbon black aggregate. A negative determination (“No” branch) at block 2170 can result in the flow of the example method 2100 being directed to optional block 2180, where the computing device can optionally determine if a termination criterion is satisfied. As is described herein, the termination criterion can be a defined number Nₘₐₓ of updates have been performed, for example. The defined number Nₘₐₓ can be defined as a multiple of the number of spheres (Nₛ (also denoted as N in some cases)) within the volume, for example—that is Nₘₐₓ=qNₛ where q is a natural number (e.g., 40, 50, or 60). Thus, after Nₘₐₓ updates (or iterations), changes to the arrangement of the defined number of spheres can cease. While not illustrated in FIG. 21, in some cases, in response to a negative determination at block 2170, the computing device can reject (or can cause the rejection of) the updated arrangement of the defined number of spheres. A negative determination (“No” branch) at optional block 2180 results in the flow of the example method 2100 being directed to block 2110, for a next update to a current arrangement of the defined number of spheres and subsequent determination of an updated value of the objective function. In scenarios where the current arrangement of the defined number of spheres has been rejected (or has been configured for rejection), the next update can be based on an immediately prior arrangement of the defined number of spheres instead of the current arrangement. An affirmative determination (“Yes” branch) at block optional 2180 results in the flow of the example method 2100 being directed to block 2190, where the computing system can configure the current model as a 3D aggregate model of the carbon black aggregate.

Although the example method 2100 is described as using a same series of binarized TEM tilt images over reconstruction steps, the disclosure is not limited in that respect. As is described herein, in some embodiments, after a defined number of reconstruction steps has been implemented, the series of binarized TEM tilt images can be updated to create another series of binarized TEM tilt images. Subsequent reconstruction steps can be implemented using that other series of binarized TEM tilt images until the defined number of reconstructions steps has again been implemented. At that point in the iterative process, that other series of binarized TEM tilt images can be updated to generate a next series of binarized TEM tilt images. Such a sequence of updates can continue until the example method 2100 ends. As is described herein, in terms of the example method 2100, a reconstruction step corresponds to the implementation of blocks 2110 to block 2160.

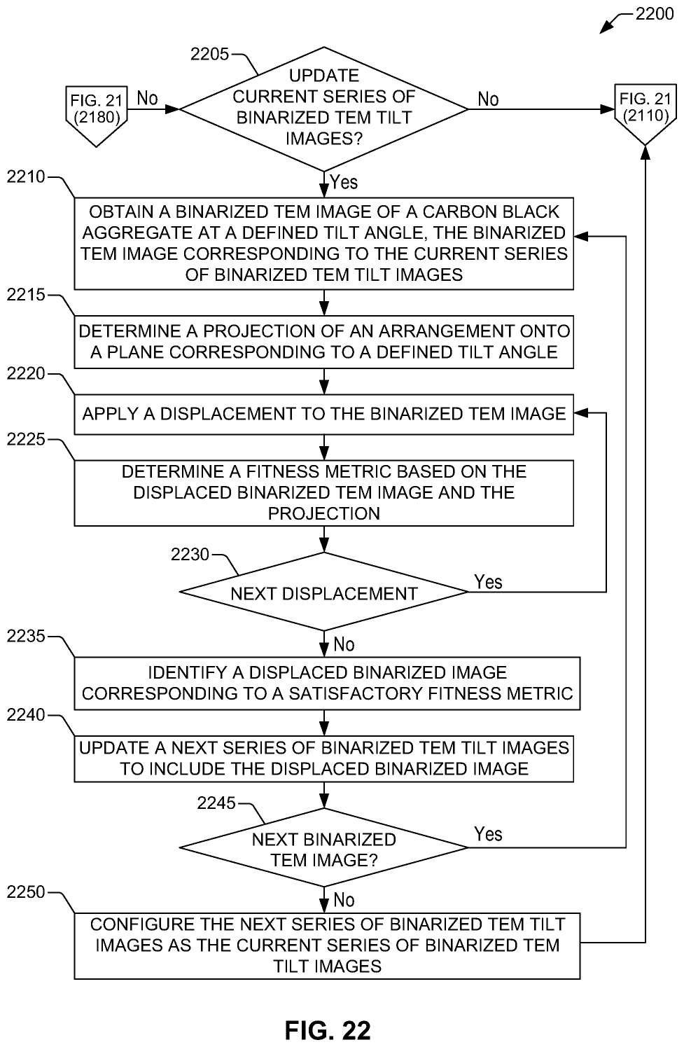

More specifically, in some embodiments, as is shown in FIG. 21, a negative determination at block 2180 can optionally direct the flow of the example method 2100 to perform the example method 2200 shown in FIG. 22. In other words, in those embodiments, before implementing a next reconstruction step, the example method 2200 can be implemented. The example method 2200 is an example of a method for updating a series of binarized TEM tilt images during, or as part of, the iterative process for generating a 3D aggregate model of a carbon black aggregate. The computing device that implements the example method 2100 (FIG. 21) also can implement the example method 2200.

At block 2205, the computing device can determine if a current series of binarized TEM tilt images of a carbon black aggregate is to be updated. As is shown in FIG. 22 and is described herein, in some cases, block 2205 can be implemented in response to a “No” branch from optional block 2180 (FIG. 21). The current series of binarized TEM tilt images corresponds to an observed series of TEM tilt images for the carbon black aggregate. In some instances, the computing device can determine that an update criterion is not satisfied and, as a result, the computing device can determine that the current series of binarized TEM tilt images is not to be updated. Such a negative determination (“No” branch) results in the flow of the example method 2200 being directed to block 2110 in the example method 2100, to implement a next reconstruction step. In other instances, the computing device can determine that the update criterion is satisfied and, as a result, the computing device can determine that current series of binarized TEM tilt images is to be updated. Such a positive determination at block 2205 (“Yes” branch) results the flow of the example method 2200 being directed to block 2210, where the computing device can obtain a binarized TEM image of the carbon black aggregate at a defined tilt angle. The binarized TEM image is part of the current series of binarized TEM tilt images and, thus, corresponds to a defined tilt angle.

At block 2215, the computing device can determine or otherwise generate a projection of an arrangement of spheres representing a current 3D model of the carbon black aggregate, as is described herein in connection with example method 2100.

At block 2220, the computing device can apply a displacement to the binarized TEM image. To that end, the displacement can be represented by a displacement vector d pertaining to a set of multiple displacement vectors. For example, each displacement vector of the set of multiple displacement vectors can be a point within a two-dimensional square lattice spanning (2N_{L}+1)×(2N_{L}+1) pixels, where N_{L} is a natural number. Such a displacement vector d can be defined in a Cartesian system of coordinates having an origin at the geometrical center of the square lattice. Thus, the displacement vector can be represented as a pair (nₓ, n_{y}), where nₓ and n_{y} are each an integer number in the interval [−N_{L},N_{L}]. The geometrical center is located at a pixel. As such, applying the displacement can include displacing the binarized TEM image by the displacement vector representing the displacement. Applying the displacement results in a displaced binarized TEM image.

At block 2225, the computing device can determine a fitness metric based on the displaced binarized TEM image and the projection. The fitness metric can be the same fitness metric described herein in connection with block 2140 in example method 2100. More specifically, the fitness metric can be the number of mismatched pixels (or, in some cases, a function of that number) resulting from a pixel-wise difference between the binarized TEM image and the projection. Thus, determining the fitness metric can include determining the number of mismatched pixels. Although not shown, as part of the example method 2200, the computing device can update a data structure to add the fitness metric that has been determined.

At block 2230, the computing device can determine if another displacement is to be applied to images in the current series of binarized TEM tilt images. In instances in which the set of multiple displacement vectors has not been traversed fully, a positive determination (“Yes” branch) can be made, and the flow of the example method 2200 can be directed to block 2220. In other instances in which the set of multiple displacement vectors has been traversed fully, a negative determination (“No” branch) is made, and the flow of the example method 2200 can be directed to block 2235, where the computing device can identify or otherwise determine a displaced binarized image corresponding to a satisfactory fitness metric. Because each fitness metric that is determined at block 2225 is indicative of a number of mismatched pixels, a satisfactory fitness metric can be indicative of the least number of mismatched pixels. That is, the displaced binarized image that is identified can have the least pixel mismatch relative to the projection.

At block 2240, the computing device can update a next series of binarized TEM tilt images to include the displaced binarized image that has been identified at block 2235. Thus, the next series of binarized TEM tilt images can be formed by displaced binarized images that each minimize the pixel mismatch with the projection determined (or otherwise generated) at block 2215.

At block 2245, the computing device can determine if a next binarized TEM image in the current series of binarized TEM tilt images is to be subjected to the set of multiple displacement vectors. In instances in which the current series of binarized TEM tilt images has not been fully traversed, a positive determination (“Yes” branch) can be made and, as a result, the flow of the example method 2200 can be directed to block 2210. In other instances in which the current series of binarized TEM tilt images has been fully traversed, a negative determination (“No” branch) can be made and, as a result, the flow of the example method 2200 is directed to block 2250, where the computing device can configure the next series of binarized TEM tilt images as the current series of binarized TEM tilt images. Flow of the example method 2200 can then be directed to block 2110 in the example method 2100 shown in FIG. 21, for implementation of further reconstructions steps.

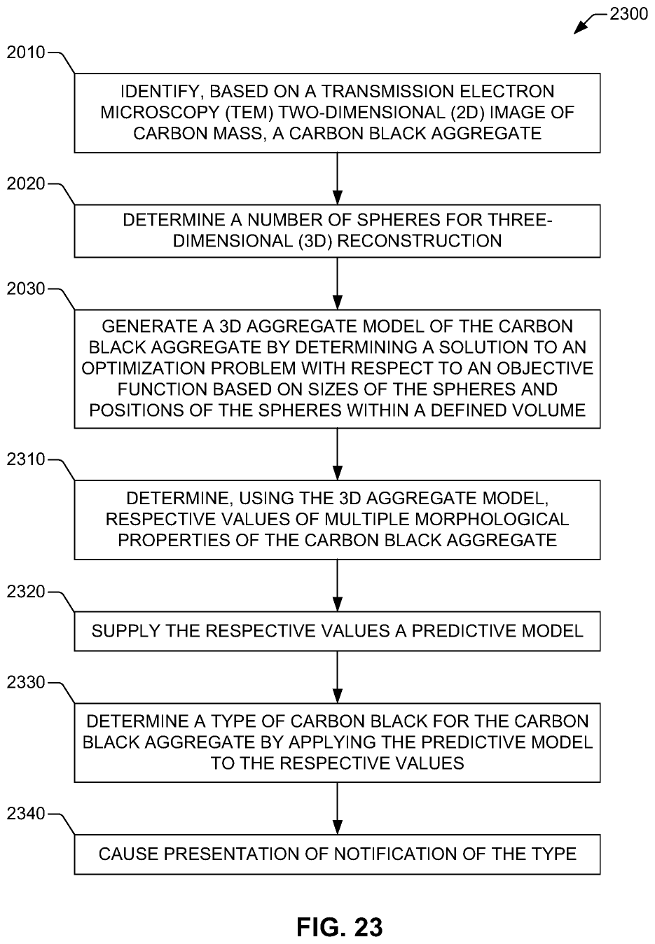

FIG. 23 illustrates an example of a method 2300 for providing a 3D reconstruction and analysis of a carbon black aggregate, in accordance with one or more embodiments of this disclosure. The example method 2300 can be implemented to identify a type of carbon black of the carbon black aggregate. A computing device or a system of computing devices can implement the example method 2300 in its entirety or in part. To that end, each one of the computing devices includes computing resources that may implement at least one of the blocks included in the example method 2300 and other methods described herein. The computing resources comprise, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. The computing device can embody the computing device 250 (FIG. 19) and, thus, can host at least one of the intake module 252, the segmentation module 256, the reconstruction module 260, the analysis module 270, the CB identification module 1910, and the reporting module 274. Accordingly, the computing device can implement the example method 2300 by executing one or more instances of such modules.

The example method 2300 includes block 2010, block 2020, and block 2030 described in connection with the example method 2000 shown in FIG. 20 and described hereinbefore. The computing device that implements the example method 2300 can implement each one of block 2010, block 2020, and block 2030 as is described hereinbefore. As a result of the implementation of block 2010, block 2020, and block 2030, a 3D aggregate model of the carbon black aggregate is generated. At block 2310, the computing device can determine, using the 3D aggregate model, respective values of multiple morphological properties of the carbon black aggregate. As mentioned, the multiple morphological properties can include aggregate anisometry metric (e.g., Zᵣₑₗ), aggregate volume, relative 3D void volume (3DVV), or 3D specific surface area (3DSSA). As part of, or in addition to, determining such values, the computing device can generate a feature vector having a number M of components that is equal to the number of multiple morphological components for which values have been determined.

At block 2320, the computing device can supply the respective values to a predictive model (e.g., predictive model 1920 (FIG. 19)). To that end, a component (e.g., analysis module 270 (FIG. 19)) of the computing device can pass the feature vector to another component (e.g., the CB identification module 1910) that has the prediction model 1920.

At block 2330, the computing device can determine a type of carbon black for the carbon black aggregate by applying the predictive model to the feature vector containing the respective values. In some cases, applying the predictive model can include applying a defined procedure corresponding to the predictive with the feature vector as argument to the procedure.

At block 2340, the computing device can cause presentation of a notification of the type of carbon black. The notification can be presented at a display device (e.g., display device 280 (FIG. 19)) functionally coupled to or integrated into the computing device.

Although not illustrated in FIG. 23, in some embodiments, rather than analyzing a carbon black aggregate individually, the example method 2300 can be implemented for a group of carbon black aggregates. To that end, blocks 2010 to 2030, blocks 2310 to 2330 can be implemented for each carbon black aggregate in the group of carbon black aggregates. The computing device can then evaluate the various predictions for respective carbon black aggregates in the group of carbon black aggregates, and based on the evaluation, the computing device can determine one or more representative types of carbon black for the group of carbon black aggregates. In some cases, the computing device can rank the one or more respective types, and can then cause presentation of a notification or indicia indicative of a ranking of the one or more respective types. The notification and/or indicia can be presented at a display device.

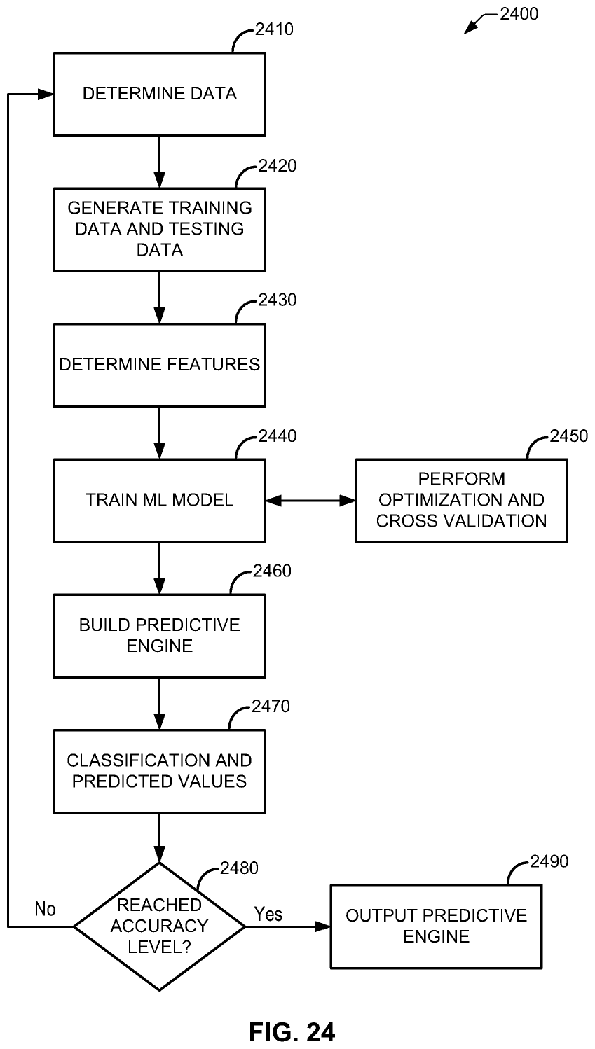

FIG. 24 illustrates an example of a training method 2400 for generating a predictive model, in accordance with one or more embodiments of this disclosure. The example training method 2400 is an example of a supervised learning method; variations of this example of training method are discussed below, however, other training methods can be analogously implemented to train unsupervised and/or semi-supervised machine learning (predictive) models. A computing device that hosts the training module 1810 can implement the example training method 2400 via the training module 1810. Such a computing device can include computing resources that may implement at least one of the blocks included in the example method 2000 and other methods described herein. The computing resources comprise, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. In some cases, such a computing device can be the computing device 250 described herein.

At block 2410, the example training method 2400 can determine information determining morphological properties of carbon black aggregates. Such information may contain one or more datasets. Each dataset may include labeled baseline data.

The example training method 2400 can generate, at block 2420, a training dataset and a testing dataset. The training dataset and the testing dataset can be generated by configuring labeled feature vectors, each including respective values of multiple morphological properties of a carbon black aggregate. In some cases, as is described herein, M morphological properties can define a feature vector. Respective values of the M morphological properties define an M-tuple that embodies an instance of a feature vector. The training dataset and the testing dataset may be generated by randomly assigning data, formatted as feature vectors, to either the training dataset or the testing dataset. In some instances, the assignment of data as training or test samples may not be completely random. In some instances, only the labeled baseline data for a specific feature extracted from data associated with morphological properties of carbon black aggregates can be used to generate the training dataset and the testing dataset. In some instances, a majority of the labeled baseline data extracted from data associated with morphological properties of carbon black aggregates can be used to generate the training dataset. For example, 75% of the labeled baseline data can form the training dataset and 25% of that labeled data can form the testing dataset. Any method or technique may be used to create the training and testing datasets.

The example training method 2400 can determine (e.g., extract, select, etc.), at block 2430, one or more features that can be used by, for example, a classifier to designate a carbon black aggregate as pertaining to a particular type of carbon black. The example training method 2400 can determine a set of training baseline features from the training dataset.

The example training method 2400 can train one or more machine learning models using the one or more features at block 2440. In some instances, the machine learning models may be trained using supervised learning. In another embodiment, other machine learning techniques may be employed, including unsupervised learning and semi-supervised. The machine learning models trained at block 2440 may be selected based on different criteria and/or data available in the training dataset. For example, machine learning classifiers can suffer from different degrees of bias. Accordingly, more than one machine learning model can be trained at block 2440, optimized, improved, and cross-validated at 2450.

The example training method 2400 can select one or more machine-learning models to build a predictive engine at 2460 (e.g., a machine learning classifier, a predictive model, etc.). The predictive engine can be evaluated using the testing dataset. The predictive engine can analyze the testing dataset and generate classification values and/or predicted values at block 2470. Classification and/or prediction values can be evaluated at block 2480 to determine whether such values have achieved a desired accuracy level. Performance of the predictive engine can be evaluated in a number of ways based on a number of true positives, false positives, true negatives, and/or false negatives classifications of the plurality of data points indicated by the predictive engine. For example, the false positives of the predictive engine can refer to a number of times the predictive engine incorrectly determined exposure and/or a risk of exposure to an airborne pathogen and/or the like. Conversely, the false negatives of the predictive engine can refer to a number of times the machine learning model determined exposure and/or a risk of exposure to an airborne pathogen and/or the like incorrectly, when in fact, the determined exposure and/or a risk of exposure to an airborne pathogen and/or the like matches an actual exposure and/or risk of exposure. True negatives and true positives may refer to a number of times the predictive engine correctly determined exposure and/or a risk of exposure to an airborne pathogen and/or the like. Related to these measurements are the concepts of recall and precision. Generally, recall refers to a ratio of true positives to a sum of true positives and false negatives, which quantifies a sensitivity of the predictive engine. Similarly, precision refers to a ratio of true positives a sum of true and false positives.

When such a desired accuracy level is reached (“Yes” branch), the training phase ends and the predictive engine can be output at 2490. When the desired accuracy level is not reached (“No” branch), then a subsequent iteration of the example training method 2400 can be performed starting at bock 2410 with variations such as, for example, considering a larger collection of data relating to morphological properties of carbon black aggregates.

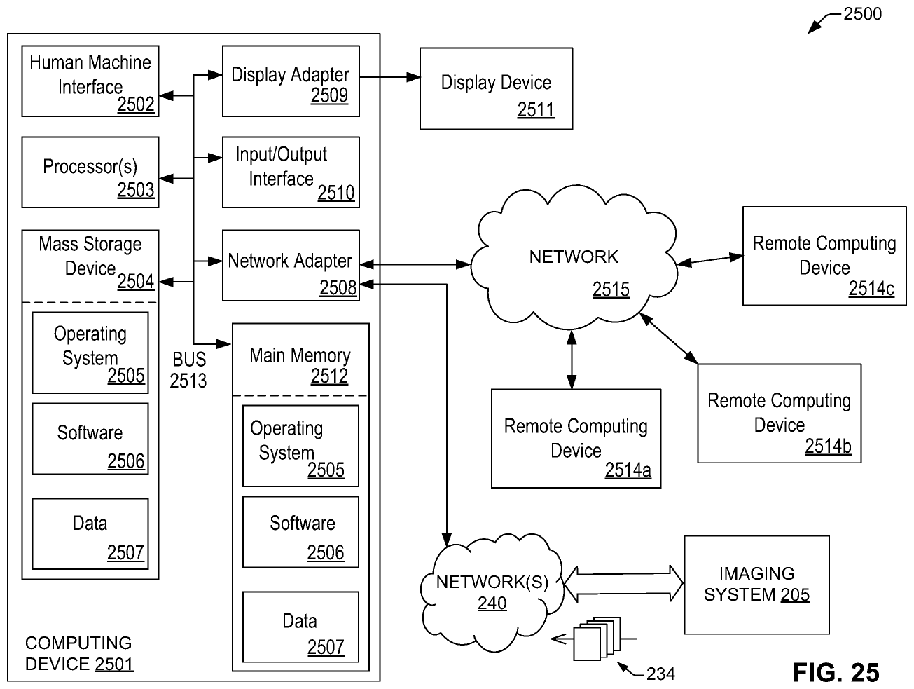

The 3D analysis of carbon black in accordance with aspects described herein can be implemented on the computing system 2500 illustrated in FIG. 25 and described below. The computer-implemented methods, devices, and systems disclosed herein may utilize one or more computing devices to perform one or more functions in one or more locations. FIG. 25 is a block diagram depicting an example computing system 2500 for performing the disclosed methods and/or implementing the disclosed systems. The computing system 2500 is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment. The computing environment 2500 shown in FIG. 25 can embody, or can constitute, the example operating environment 200 (FIG. 2). The computing system 2500 may implement the various functionalities described herein in connection with TEM-based 3D reconstruction and analysis of carbon black aggregates. For example, one or more of the computing devices that form the computing system 2500 can include intake module 252, segmentation module 256, reconstruction module 260, analysis module 270, and reporting module 274. In addition, or in some embodiments, as is described herein, the one or more computing devices that form the computing system 2500 also can optionally include the CB identification module 1910. Further, or in other embodiments, the one or more computing devices that from the computing system 2500 also optionally include the training module 1810.

The computer-implemented methods, devices, and systems in accordance with this disclosure may be operational with numerous other general purpose or special purpose computing system environments or configurations. Examples of well-known computing systems, environments, and/or configurations that may be suitable for use with the systems and methods comprise, but are not limited to, personal computers, server computers, laptop devices, and multiprocessor systems. Additional examples comprise set-top boxes, programmable consumer electronics, network PCs, minicomputers, mainframe computers, distributed computing environments that comprise any of the above systems or devices, and the like.

The processing of the disclosed computer-implemented methods, devices, and systems may be performed by software components. The disclosed systems, devices, and computer-implemented methods may be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods may also be practiced in grid-based and distributed computing environments where tasks are performed by remote processing devices that are linked through a communications network. In a distributed computing environment, program modules may be located in both local and remote computer storage media including memory storage devices.

Further, the systems, devices, and computer-implemented methods disclosed herein may be implemented via a general-purpose computing device in the form of a computing device 2501. The components of the computing device 2501 may comprise one or more processors 2503, a main memory 2512, and a system bus 2513 that couples various system components including the one or more processors 2503 to the main memory 2512. The system may utilize parallel computing. The computing device 2501 can be embody the computing device 250 described herein.

The system bus 2513 represents one or more of several possible types of bus structures, including a memory bus or memory controller, a peripheral bus, an accelerated graphics port, or local bus using any of a variety of bus architectures. The system bus 2513, and all buses specified in this description may also be implemented over a wired or wireless network connection and each of the subsystems, including the one or more processors 2503, a mass storage device 2504, an operating system 2505, software 2506, data 2507, a network adapter 2508, the system memory 2512, an Input/Output interface 2510, a display adapter 2509, a display device 2511, and a human-machine interface 2502, may be contained within one or more remote computing devices 2514a,b,c at physically separate locations, connected through buses of this form, in effect implementing a fully distributed system.

The computing device 2501 typically comprises a variety of computer-readable media. Exemplary readable media may be any available media that is accessible by the computing device 2501 and comprises, for example, both volatile and non-volatile media, removable and non-removable media. The main memory 2512 comprises computer readable media in the form of volatile memory, such as random access memory (RAM), and/or non-volatile memory, such as read only memory (ROM). The main memory 2512 typically contains data such as the data 2507 and/or program modules such as the operating system 2505 and the software 2506 that are immediately accessible to and/or are presently operated on by the one or more processors 2503. For example, the software 2506 may include the intake module 252, segmentation module 256, reconstruction module 260, analysis module 270, and reporting module 274. In addition, or other embodiments, the software 2506 can include the CB identification module 1910 or the training module 1810, or both. The operating system 2505 may be embodied in one of Windows operating system, Unix, or Linux, for example.

In another aspect, the computing device 2501 may also comprise other removable/non-removable, volatile/non-volatile computer storage media. For example, FIG. 25 illustrates the mass storage device 2504 which may provide non-volatile storage of computer code, computer readable instructions, data structures, program modules, and other data for the computing device 2501. For example and not meant to be limiting, the mass storage device 2504 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 2504, including by way of example, the operating system 2505 and the software 2506. Each of the operating system 2505 and the software 2506 (or some combination thereof) may comprise elements of the programming and the software 2506. The data 2507 may also be stored on the mass storage device 2504. The data 2507 may be stored in any of one or more databases known in the art. Examples of such databases comprise, DB2®, Microsoft® Access, Microsoft® SQL Server, Oracle®, mySQL, PostgreSQL, SQLite, and the like. The databases may be centralized or distributed across multiple systems.

In another aspect, the user may enter commands and information into the computing device 2501 via an input device (not shown). Examples of such input devices comprise, but are not limited to, a keyboard, pointing device (e.g., a “mouse”), a microphone, a joystick, a scanner, tactile input devices such as gloves, and other body coverings, and the like. These and other input devices may be connected to the one or more processors 2503 via the human-machine interface 2502 that is coupled to the system bus 2513, but may be connected by other interface and bus structures, such as a parallel port, game port, an IEEE 1394 Port (also known as a Firewire port), a serial port, or a universal serial bus (USB).

In yet another aspect, the display device 2511 may also be connected to the system bus 2513 via an interface, such as the display adapter 2509. It is contemplated that the computing device 2501 may have more than one display adapter 2509 and the computing device 2501 may have more than one display device 2511. For example, the display device 2511 may be a monitor, an LCD (Liquid Crystal Display), or a projector. In addition to the display device 2511, other output peripheral devices may comprise components such as speakers (not shown) and a printer (not shown) which may be connected to the computing device 2501 via the Input/Output Interface 2510. Any operation and/or result of the methods may be output in any form to an output device. Such output may be any form of visual representation, including, but not limited to, textual, graphical, animation, audio, tactile, and the like. The display device 2511 and computing device 2501 may be part of one device, or separate devices.

The computing device 2501 may operate in a networked environment using logical connections to one or more remote computing devices 2514a,b,c. For example, a remote computing device may be a personal computer, portable computer, smartphone, a server device, a router device, a network computer, a peer device or other common network node, and so on. Logical connections between the computing device 2501 and a remote computing device 2514a,b,c may be made via a network 2515, such as a LAN and/or a general WAN. Such network connections may be through the network adapter 2508. The network adapter 2508 may be implemented in both wired and wireless environments. The network 2515 may embody, or can constitute, the network(s) 240 (FIG. 2), for example.

For purposes of illustration, application programs and other executable program components such as the operating system 2505 are illustrated herein as discrete blocks, although it is recognized that such programs and components reside at various times in different storage components of the computing device 2501, and are executed by the one or more processors 2503 of the computer. An implementation of the software 2506 may be stored on or transmitted across some form of computer-readable media. Any of the disclosed methods may be performed by computer readable instructions embodied on computer-readable media. Computer-readable media may be any available media that may be accessed by a computer. By way of example and not meant to be limiting, computer-readable media may comprise “computer storage media” and “communications media.” “Computer storage media” comprise volatile and non-volatile, removable and non-removable media implemented in any methods or technology for storage of information such as computer-readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer.

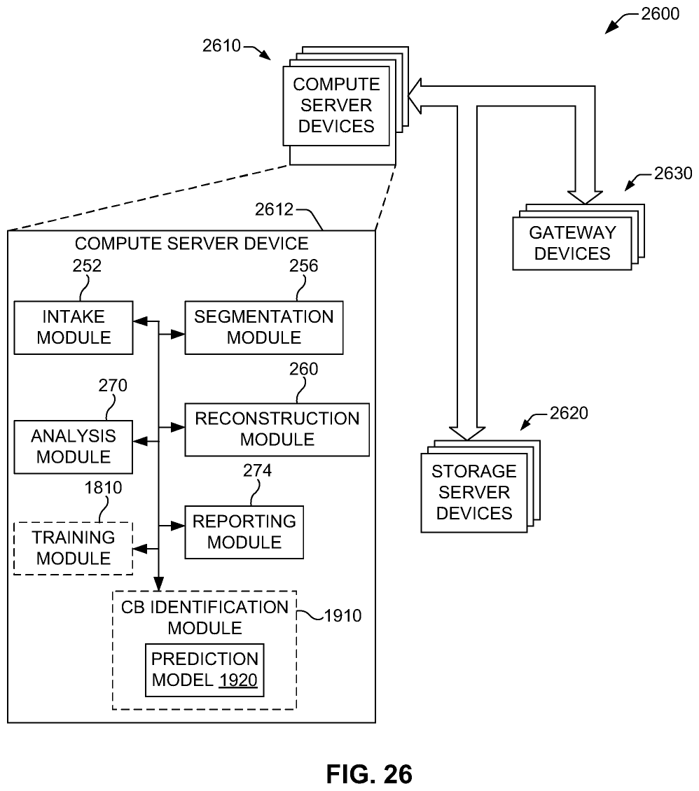

FIG. 26 is a schematic block diagram of an example of a computing system 2600 that can implement 3D analysis of carbon black, in accordance with one or more embodiments of this disclosure. The computing system 2600 that can embody, or can include, the modeling system 245 included in the operating environment 200 (FIG. 2) or the operating environment 1900 (FIG. 19). Accordingly, the example computing system 2600 can provide the functionality described herein in connection with TEM-based 3D reconstruction and analysis of carbon black aggregates implemented in the operating environment 200 and described herein.

The example computing system 2600 includes two types of server devices: Compute server devices 2610 and storage server devices 2620. A subset of the compute server devices 2610, individually or collectively, can host the various modules that permit implementing TEM-based 3D reconstruction and analysis of carbon black aggregates in accordance with aspects described herein. Thus, such a subset of compute server device 2610 can operate in accordance with functionality described herein in connection with TEM-based 3D reconstruction and analysis of carbon black aggregates. For the sake of illustration, a compute server device 2612 within such a subset is schematically depicted as hosting those modules—e.g., intake module 252, segmentation module 256, reconstruction module 260, analysis module 270, and reporting module 274. The compute server device 2612 also can optionally include the CB identification module 1910 (FIG. 19) or the training module 1810 (FIG. 18), or both. Similar to the computing device 2401 (FIG. 24), the architecture of the compute server device 2612 comprises one or more processors, one or more memory devices, and a bus architecture that functionally couples the processor(s) and the memory device(s). In some cases, the modules hosted by the compute server device 2612 can be stored in at least one of such memory device(s). At least the subset of the compute server devices 2610 can be functionally coupled to one or multiple ones of the storage server devices 2620. That coupling can be direct or can be mediated by at least one of gateway devices 2630. The storage server devices 2620 include data and/or metadata that can be used to implement the functionality described herein in connection with TEM-based 3D reconstruction and analysis of carbon black aggregates. Some or all of the storage server devices 2620 can embody, or can constitute, the memory 278. In addition, or in some embodiments, some or all of the storage server devices can embody, or can constitute, the labeled data repository 1804 and the model repository 1830.

Each one of the gateway devices 2630 can include one or multiple processors functionally coupled to one or multiple memory devices that can retain application programming interfaces (APIs) and/or other types of program code for access to the compute server devices 2610 and storage server devices 2620. Such access can be programmatic, via an appropriate function call, for example. The subset of the compute server devices 2610 that host one or a combination of the intake module 252, segmentation module 256, reconstruction module 260, analysis module 270, and reporting module 274 also can use API(s) supplied by the gateway devices 2630 in order to provide results of implementing the functionalities described herein in connection with TEM-based 3D reconstruction and analysis of carbon black aggregates in accordance with aspects described herein.

It is to be understood that the methods and systems described here are not limited to specific operations, processes, components, or structure described, or to the order or particular combination of such operations or components as described. It is also to be understood that the terminology used herein is for the purpose of describing example embodiments only and is not intended to be restrictive or limiting.

As used herein the singular forms “a,” “an,” and “the” include both singular and plural referents unless the context clearly dictates otherwise. Values expressed as approximations, by use of antecedents such as “about” or “approximately,” shall include reasonable variations from the referenced values. If such approximate values are included with ranges, not only are the endpoints considered approximations, the magnitude of the range shall also be considered an approximation. Lists are to be considered exemplary and not restricted or limited to the elements comprising the list or to the order in which the elements have been listed unless the context clearly dictates otherwise.

Throughout the specification and claims of this disclosure, the following words have the meaning that is set forth: “comprise” and variations of the word, such as “comprising” and “comprises,” mean including but not limited to, and are not intended to exclude, for example, other additives, components, integers, or operations. “Include” and variations of the word, such as “including” are not intended to mean something that is restricted or limited to what is indicated as being included, or to exclude what is not indicated. “May” means something that is permissive but not restrictive or limiting. “Optional” or “optionally” means something that may or may not be included without changing the result or what is being described. “Prefer” and variations of the word such as “preferred” or “preferably” mean something that is exemplary and more ideal, but not required. “Such as” means something that serves simply as an example.

Operations and components described herein as being used to perform the disclosed methods and construct the disclosed systems are illustrative unless the context clearly dictates otherwise. It is to be understood that when combinations, subsets, interactions, groups, etc. of these operations and components are disclosed, that while specific reference of each various individual and collective combinations and permutation of these may not be explicitly disclosed, each is specifically contemplated and described herein, for all methods and systems. This applies to all aspects of this application including, but not limited to, operations in disclosed methods and/or the components disclosed in the systems. Thus, if there are a variety of additional operations that may be performed or components that may be added, it is understood that each of these additional operations may be performed and components added with any specific embodiment or combination of embodiments of the disclosed systems and methods.

Embodiments of this disclosure may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof, whether internal, networked, or cloud-based.

Embodiments of this disclosure have been described with reference to diagrams, flowcharts, and other illustrations of computer-implemented methods, systems, apparatuses, and computer program products. Each block of the block diagrams and flowchart illustrations, and combinations of blocks in the block diagrams and flowchart illustrations, respectively, may be implemented by processor-accessible instructions. Such instructions may include, for example, computer program instructions (e.g., processor-readable and/or processor-executable instructions). The processor-accessible instructions may be built (e.g., linked and compiled) and retained in processor-executable form in one or multiple memory devices or one or many other processor-accessible non-transitory storage media. These computer program instructions (built or otherwise) may be loaded onto a general-purpose computer, special purpose computer, or other programmable data processing apparatus to produce a machine. The loaded computer program instructions may be accessed and executed by one or multiple processors or other types of processing circuitry. In response to execution, the loaded computer program instructions provide the functionality described in connection with flowchart blocks (individually or in a particular combination) or blocks in block diagrams (individually or in a particular combination). Thus, such instructions which execute on the computer or other programmable data processing apparatus create a means for implementing the functions specified in the flowchart blocks (individually or in a particular combination) or blocks in block diagrams (individually or in a particular combination).

These computer program instructions may also be stored in a computer-readable memory that may direct a computer or other programmable data processing apparatus to function in a particular manner, such that the instructions stored in the computer-readable memory produce an article of manufacture including processor-accessible instruction (e.g., processor-readable instructions and/or processor-executable instructions) to implement the function specified in the flowchart blocks (individually or in a particular combination) or blocks in block diagrams (individually or in a particular combination). The computer program instructions (built or otherwise) may also be loaded onto a computer or other programmable data processing apparatus to cause a series of operations to be performed on the computer or other programmable apparatus to produce a computer-implemented process. The series of operations may be performed in response to execution by one or more processor or other types of processing circuitry. Thus, such instructions that execute on the computer or other programmable apparatus provide operations for implementing the functions specified in the flowchart blocks (individually or in a particular combination) or blocks in block diagrams (individually or in a particular combination).

Accordingly, blocks of the block diagrams and flowchart diagrams support combinations of means for performing the specified functions in connection with such diagrams and/or flowchart illustrations, combinations of operations for performing the specified functions and program instruction means for performing the specified functions. Each block of the block diagrams and flowchart illustrations, and combinations of blocks in the block diagrams and flowchart illustrations, may be implemented by special purpose hardware-based computer systems that perform the specified functions or operations, or combinations of special purpose hardware and computer instructions.

As is used in this specification and annexed drawings, the terms “module,” “component,” “system,” “platform,” and the like, can refer to and/or can include a computer-related entity or an entity related to an operational machine with one or more specific functionalities. Such entities can be either hardware, a combination of hardware and software, software (program code or executable program code, for example), or software in execution. In one example, a component can be a process running on a processor, a processor, an object, an executable (e.g., binary software), a thread of execution, a computer program, and/or a computing device. Simply as an illustration, a software application running on a server device can be a component and the server device also can be a component. One or more modules can reside within a process and/or thread of execution. One or more components also can reside within a process and/or thread of execution. Each one of a module and a component can be localized on one computing device and/or distributed between two or more computing devices. In another example, respective components (or modules) can execute from various computer-readable storage media having various data structures stored thereon. The components (or modules) can communicate via local and/or remote processes such as in accordance with a signal having one or more data packets (e.g., data from one component interacting with another component in a local system, distributed system, and/or across a network such as the Internet with other systems via the signal). As another illustrations, in some cases, a component can emulate an electronic component via a virtual machine, e.g., within a cloud computing system. The terms “module” and “component” (and their plural versions) may be used interchangeably where clear from context, in some cases.

As is used in this specification and annexed drawings, the term “processor” can refer to substantially any computing processing unit or computing device, including single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to electronic circuitry designed in assembled to execute code instructions and/or operate on data and signaling. Such electronic circuitry can be assembled in a chipset, for example. Accordingly, in some cases, a processor can be embodied, or can include, an application specific integrated circuit (ASIC), a digital signal processor (DSP), a field programmable gate array (FPGA), a complex programmable logic device (CPLD), a discrete gate or transistor logic, discrete hardware components, or any combination thereof designed and assembled to perform the functionality described herein. Further, in some cases, processors can exploit nano-scale architectures, such as molecular and quantum-dot based transistors, switches and gates, in order to optimize space usage or enhance performance of computing devices. A processor can also be implemented as a combination of computing processing units.

Further, in this specification and annexed drawings, terms such as “storage,” “data storage,” “repository,” and substantially any other information storage component relevant to operation and functionality of a system, subsystem, module, and component are utilized to refer to “memory components,” entities embodied in a “memory,” or components including a memory. As is described herein, memory and/or memory components of this disclosure can be either volatile memory or nonvolatile memory, or can include both volatile and nonvolatile memory. Simply as an illustration, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Embodiments of this disclosure are not limited to these types of memory, and other types of memory devices can be contemplated.

The methods and systems may employ artificial intelligence techniques such as machine learning and iterative learning. Examples of such techniques include, but are not limited to, expert systems, case-based reasoning, Bayesian networks, behavior-based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

While the computer-implemented methods, apparatuses, devices, and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its operations be performed in a specific order. Accordingly, where a method claim does not actually recite an order to be followed by its operations or it is not otherwise specifically stated in the claims or descriptions that the operations are to be limited to a specific order, it is in no way intended that an order be inferred, in any respect. This holds for any possible non-express basis for interpretation, including: matters of logic with respect to arrangement of operations or operational flow; plain meaning derived from grammatical organization or punctuation; the number or type of embodiments described in the specification.

It will be apparent to those skilled in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A computer-implemented method comprising:
identifying, using a two-dimensional (2D) transmission electron microscopy (TEM) image, a carbon black aggregate;
determining, based at least on an approximation to a volume of the carbon black aggregate, a number of spheres; and
generating a three-dimensional (3D) aggregate model of the carbon black aggregate by determining a solution to an optimization problem with respect to an objective function based on sizes of the spheres and positions of the spheres within a defined volume, the solution representing the 3D aggregate model.

2. The computer-implemented method of claim 1, further comprising causing presentation of a notification that the 3D aggregate model is available for analysis.

3. The computer-implemented method of claim 1, further comprising,
determining, using the 3D aggregate model, respective values of one or more morphological properties of the carbon black aggregate; and
supplying the respective values of the one or more morphological properties.

4. The computer-implemented method of claim 3, wherein the one or more morphological properties comprise at least one of aggregate anisometry metric, aggregate volume, relative 3D void volume (3DVV), or 3D specific surface area (3DSSA).

5. The computer-implemented method of claim 3, wherein the supplying comprises,
retaining data indicative of the one or more morphological properties; and
configuring an application programming interface to access a value of a first morphological property of the one or more morphological properties.

6. The computer-implemented method of claim 1, wherein the determining the solution to the optimization problem comprises,
updating a current arrangement of the spheres, wherein the current arrangement of spheres comprises a distribution of sizes of the spheres and a distribution of positions of the spheres within the defined volume; and
determining multiple 2D projections of the configuration of the spheres on respective defined planes.

7. The computer-implemented method of claim 6, wherein the updating comprises at least one of,
modifying a position of a first sphere of the spheres and maintaining second positions of second spheres of the spheres; or
modifying a size of the first sphere and maintaining second sizes of the second spheres.

8. The computer-implemented method of claim 6, wherein the determining the solution to the optimization problem further comprises determining multiple fitness metrics for respective ones of the multiple 2D projections, each fitness metric of the multiple fitness metrics being based on a respective binarized 2D image of the carbon black aggregate.

9. The computer-implemented method of claim 8, wherein the determining the solution to the optimization problem further comprises updating the objective function based on an average of the multiple fitness metrics.

10. The computer-implemented method of claim 9, further comprising,
determining that the objective function satisfies a convergence criterion; and
configuring the current arrangement of spheres as the 3D aggregate model.

11. A computing device comprising:
one or more processors; and
one or more memory devices storing computer-executable instructions that, in response to execution by the one or more processors, cause the computing device to,
identify, using a two-dimensional (2D) transmission electron microscopy (TEM) image, a carbon black aggregate;
determine, based at least on an approximation to a volume of the carbon black aggregate, a number of spheres; and
generate a three-dimensional (3D) aggregate model of the carbon black aggregate by determining a solution to an optimization problem with respect to an objective function based on sizes of the spheres and positions of the spheres within a defined volume, the solution representing the 3D aggregate model.

12. The computing device of claim 11, the one or more memory devices storing further computer-executable instructions that, in response to execution by the one or more processors, further cause the computing device to cause presentation of a notification that the 3D aggregate model is available for analysis.

13. The computing device of claim 11, the one or more memory devices storing further computer-executable instructions that, in response to execution by the one or more processors, further cause the computing device to,
determine, using the 3D aggregate model, respective values of one or more morphological properties of the carbon black aggregate; and
supply the respective values of the one or more morphological properties.

14. The computing device of claim 11, wherein the determining the solution to the optimization problem comprises,
updating a current arrangement of the spheres, wherein the current arrangement of spheres comprises a distribution of sizes of the spheres and a distribution of positions of the spheres within the defined volume; and
determining multiple 2D projections of the configuration of the spheres on respective defined planes.

15. The computing device of claim 14, wherein the determining the solution to the optimization problem further comprises determining multiple fitness metrics for respective ones of the multiple 2D projections, each fitness metric of the multiple fitness metrics being based on a respective binarized 2D image of the carbon black aggregate.

16. The computing device of claim 15, wherein the determining the solution to the optimization problem further comprises updating the objective function based on an average of the multiple fitness metrics.

17. The computing device of claim 16, the one or more memory devices storing further computer-executable instructions that, in response to execution by the one or more processors, further cause the computing device to, determine that the objective function satisfies a convergence criterion; and
configure the current arrangement of spheres as the 3D aggregate model.

18. At least one non-transitory computer-readable storage medium having processor-executable instructions encoded thereon that, in response to execution, cause a computing device to,
identify, using a two-dimensional (2D) transmission electron microscopy (TEM) image, a carbon black aggregate;
determine, based at least on an approximation to a volume of the carbon black aggregate, a number of spheres; and
generate a three-dimensional (3D) aggregate model of the carbon black aggregate by determining a solution to an optimization problem with respect to an objective function based on sizes of the spheres and positions of the spheres within a defined volume, the solution representing the 3D aggregate model.

19. The at least one non-transitory computer-readable storage medium of claim 18, wherein the processor-executable instructions, in response to further execution, further cause the computing device to cause presentation of a notification that the 3D aggregate model is available for analysis.

20. The at least one non-transitory computer-readable storage medium of claim 18, wherein the processor-executable instructions, in response to further execution, further cause the computing device to,
determine, using the 3D aggregate model, respective values of one or more morphological properties of the carbon black aggregate; and
supply the respective values of the one or more morphological properties.

21. The at least one non-transitory computer-readable storage medium of claim 18, wherein the determining the solution to the optimization problem comprises,
updating a current arrangement of the spheres, wherein the current arrangement of spheres comprises a distribution of sizes of the spheres and a distribution of positions of the spheres within the defined volume; and
determining multiple 2D projections of the configuration of the spheres on respective defined planes.

22. The at least one non-transitory computer-readable storage medium of claim 21, wherein the determining the solution to the optimization problem further comprises determining multiple fitness metrics for respective ones of the multiple 2D projections, each fitness metric of the multiple fitness metrics being based on a respective binarized 2D image of the carbon black aggregate.

23. The at least one non-transitory computer-readable storage medium of claim 22, wherein the determining the solution to the optimization problem further comprises updating the objective function based on an average of the multiple fitness metrics.

24. The at least one non-transitory computer-readable storage medium of claim 23, the one or more memory devices storing further computer-executable instructions that, in
response to execution by the one or more processors, further cause the computing device to,
determine that the objective function satisfies a convergence criterion; and
configure the current arrangement of spheres as the 3D aggregate model.
